(19) United States
(12) Patent Application Publication
(10) Pub. No.: US 2023/0381519 A1
(43) Pub. Date: Nov. 30, 2023

(54) NEUROMODULATION OF BARORECEPTOR REFLEX

(71) Applicants: CASE WESTERN RESERVE UNIVERSITY, Cleveland, OH (US); Galvani Bioelectronics Limited, Brentford (GB)

(72) Inventors: Arun SRIDHAR, Brentford (GB); Stephen J. LEWIS, Cleveland, OH (US); Ibrahim SALMAN, Cleveland, OH (US)

(73) Assignees: CASE WESTERN RESERVE UNIVERSITY, Cleveland, OH (US); Galvani Bioelectronics Limited, Brentford (GB)

(21) Appl. No.: 18/158,563

(22) Filed: Jan. 24, 2023

Related U.S. Application Data
(63) Continuation of application No. 16/643,877, filed on Mar. 3, 2020, now abandoned.
(60) Provisional application No. 62/558,547, filed on Sep. 14, 2017.
(60) Provisional application No. 62/667,265, filed on May 4, 2018.

(51) Int. Cl. A61N 1/36

(52) U.S. Cl. CPC A61N 1/36139; A61N 1/36117; A61N 1/36157; A61N 1/36175; A61N 1/36178

(57) ABSTRACT

Modulation of neural activity of a subject's aortic depressor nerve (ADN) and/or carotid sinus nerve (CSN) can modulate baroreceptor reflex function, thereby providing ways of treating or preventing disorders associated with malfunction or loss of the baroreceptor reflex.

### CROSS-REFERENCE TO RELATED APPLICATION

This application is a continuation of U.S. patent application Ser. No. 16/643,877, filed on Mar. 3, 2020 which is a national stage application of PCT/GB2018/052617, filed on Sep. 14, 2018 and claims priority from U.S. Patent Application No. 62/667,265 filed on May 4, 2018 and U.S. Patent Application No. 62/558,547, filed on Sep. 14, 2017. Each of these applications and the entire subject matter of each of the applications is incorporated herein by reference.

### TECHNICAL FIELD

This present disclosure relates to neuromodulation of the baroreceptor reflex, and medical devices and systems for neuromodulation of the baroreceptor reflex. The present disclosure also relates to treatment and prevention of disorders associated with the malfunction or loss of the baroreceptor reflex.

### BACKGROUND ART

The arterial baroreceptor reflex is a vital regulatory mechanism that is primarily responsible for the maintenance of arterial blood pressure in a relatively narrow range of oscillation [1,2,3,4,5,6,7,8]. The arterial baroreflex acts by reciprocal modulation of the sympathetic and parasympathetic activities that control heart rate (HR) and vascularresistance.

The loss of baroreceptor reflex function promotes development of hypertension and arterial blood pressure lability at rest [9,10,11,12,13,14,15,16], and a variety of clinically important conditions such as cardiac arrhythmias, poor cerebral perfusion that contributes to the expression of vascular dementias, and exacerbated changes in arterial blood pressure and heart rate during sleep and arousal.

Previous reports demonstrated that electrical stimulation of the baroreceptor afferent nerves elicited cardiovascular responses and hemodynamic responses [17,18,19,20,21,22,23,24]. However, these studies stimulated the baroreceptor nerves with high intensity signals, for example, reference 23 used large current amplitudes or voltages (1 mA), long pulse width (2 ms) or high frequencies (90 Hz). The neuromodulation methods in these studies are energy inefficient and are not ideal for therapeutic purposes.

### SUMMARY

The present disclosure aims to provide further and improved ways to treat disorders by modulating baroreceptor reflex function. In particular, the present disclosure aims to provide further and improved ways to treat and prevent disorders associated with the malfunction or loss of the baroreceptor reflex.

The inventors found that reversible modulation (e.g. stimulation) of the neural activity of the baroreceptor afferent fibers is capable of modulating the baroreceptor reflex, therefore providing a useful way of restoring the body's homeostatic mechanisms, such as the cardiovascular system (e.g. maintaining blood pressure at nearly constant levels), the respiratory system and the pain regulatory system. Hence, the present disclosure is useful for treating and preventing disorders associated with the malfunction or loss of the baroreceptor reflex, such as cardiovascular disorders and disorders associated therewith, and cardiorespiratory disorders and disorders associated therewith.

An aspect of the present disclosure involves reversible modulation (e.g. stimulation) of a subject's aortic depressor nerve (ADN) for treating and preventing disorders associated with the malfunction or loss of the baroreceptor reflex. The inventors found that reversible electrical stimulation of the ADN resulted in the reduction in the mean arterial blood pressure, reduction in heart rate, increase in minute ventilation and reduction in disordered breathing index in spontaneously hypertensive rats (see examples). These responses are particularly effective with low intensity, intermittent electrical signals (see examples). In certain embodiments, the responses may be particularly effective when the left ADN is reversibly modulated. The left ADN may be unilaterally modulated. The inventors have found that the unilateral reversible modulation of the left ADN may be particularly effective for eliciting decreased heart rate and decreased vascular resistance, evoking greater depressor responses. The modulation of the left ADN may be particularly effective for evoking greater depressor responses in normotensive and hypertensive males and normotensive female subjects.

In certain embodiments, the inventors found that reversible electrical stimulation of the ADN elicits a significant decrease in heart rate.

Another aspect of the present disclosure involves reversible modulation (e.g. stimulation) of a subject's carotid sinus nerve (CSN) for treating and preventing disorders associated with the malfunction or loss of the baroreceptor reflex. The inventors found that reversible electrical stimulation of the CSN resulted in the reduction in the mean arterial blood pressure and reduction in heart rate in spontaneously hypertensive rats (see examples). Furthermore, the effects produced by modulating the neural activity of the ADN can be extrapolated to modulation of the neural activity of the CSN because the ADN and the CSN have similar function and are similar in size.

A further aspect of the present disclose involves reversible modulation (e.g. stimulation) of a subject's aortic depressor nerve (ADN) and carotid sinus nerve (CSN) for treating and preventing disorders associated with the malfunction or loss of the baroreceptor reflex. Modulation (e.g. stimulation) of the neural activity of both the ADN and CSN would be particularly effective because of their cooperativity, especially between ipsilateral ADN and CSNafferents.

Thus, the present disclosure provides a system for modulating neural activity in a subject's ADN and/or CSN, the system comprising: at least one neural interfacing element having at least one electrode arranged to be in signaling contact with the nerve, and at least one voltage or current source arranged to generate at least one signal to be applied to the nerve via the at least one electrode to modulate the neural activity of the nerve to produce a change in a physiological parameter in the subject, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In certain embodiments, wherein the system is for modulating neural activity in a subject's ADN, the system may be particularly effective in producing a decrease in heart rate. In certain embodiments, wherein the system is for modulating neural activity in a subject's ADN, the system may be particularly effective in decreasing mesenteric vascular resistance. In certain embodiments, wherein the system is for modulating neural activity in a hypertensive male or normotensive male or female subject's ADN, the system is particularly effective in decreasing femoral vascular resistance. In certain embodiments, wherein the system is for modulating the neural activity in a normotensive female subject's ADN, the system may elicit a biphasic response in femoral vascular resistance (FVR), for example, the system may elicit an initial decrease in FVR followed by an increase in FVR.

The use of a low intensity signal (i.e. where the total intensity of the signal received by the nerve is below the predetermined threshold as defined herein, or an intermittent signal with a predetermined duty cycle as described herein) is particularly advantageous because the baroreceptor reflex system is tightly regulated, and so the use of a high intensity signal such as in the devices and systems in the art to modulate (e.g. stimulate) the baroreceptor afferent nerves (i.e. where the total intensity of the signal received by the nerve is above the predetermined threshold as defined herein) is likely to trigger compensatory mechanisms, which would result in reduced efficacy of CSN processing. In contrast, the use of a low intensity signal to modulate (e.g. stimulate) the baroreceptor afferent nerves is likely to allow the baroceptor reflex system to adapt in a positive way, in accordance with the present disclosure. For example, the threshold value of the present disclosure may be ≤0.03 mAs. In contrast, Reference 23 used a high intensity signal, namely between 0.05 mAs to 0.9 mAs (1 mA, pulse width 2 ms, 5 Hz-90 Hz for 5 seconds).

The present disclosure also provides a system as described herein, comprising a detector (e.g. physiological sensor subsystem) configured to: detect one or more signals indicative of one or more physiological parameters; determine from the one or more signals one or more physiological parameters; determine the one or more physiological parameters indicative of worsening of the physiological parameter; and cause the signal to be applied to the ADN and/or CSN via the at least neural interfacing element, wherein the physiological parameter is one or more of the group consisting of: systemic arterial blood pressure (systolic pressure, diastolic pressure, or mean arterial pressure), heart rate, heart rhythm, electrical conduction in the heart and heart contractility (e.g. ventricular pressure, ventricular contractility, activation-recovery interval, effective refractory period, stroke volume, ejection fraction, end diastolic fraction, stroke work, arterial elastance), vascular resistance (e.g. total peripheral resistance, mesenteric vascular resistance or femoral vascular resistance), cardiac output, rate of blood flow (e.g. systemic blood flow, or cerebral blood flow), minute ventilation, and pain perception. In one aspect, the system may comprise a processor for determining the total intensity received by the nerve from the signal. In a further aspect, the processor adjusts one or more of the signal parameters such that the total intensity received by the nerve from the signal is below the predetermined threshold.

The present disclosure also provides a method of treating or preventing a disorder associated with malfunction or loss of the baroreceptor reflex in a subject by reversibly modulating neural activity of a subject's ADN and/or CSN, comprising: (i) implanting in the subject a system of the present disclosure; positioning the neural interfacing element in signaling contact with the ADN and/or CSN; and optionally (iii) activating the system.

Similarly, the present disclosure provides a method of reversibly modulating (e.g. stimulating) neural activity of a subject's ADN and/or CSN, comprising: (i) implanting in the subject a system of the present disclosure; (ii) positioning the neural interfacing element of the system in signaling contact with the nerve; and optionally (iii) activating the system.

The present disclosure also provides a method of implanting a system of the present disclosure in a subject, comprising: positioning a neural interfacing element of the system in signaling contact with the subject's ADN and/or CSN.

The present disclosure also provides a method for treating or preventing a disorder associated with malfunction or loss of the baroreceptor reflex, comprising: applying a signal to a subject's ADN and/or CSN via at least one neural interfacing element having at least one electrode in signaling contact with the ADN and/or CSN, such that the signal reversibly modulates neural activity of the ADN and/or CSN to produce a change in a physiological parameter in the subject, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In certain embodiments, wherein the method for treating or preventing a disorder comprises applying a signal to a subject's ADN, the method may be particularly effective in producing a decrease in heart rate.

In certain embodiments, wherein the method for treating or preventing a disorder comprises applying a signal to a subject's ADN, the method may be particularly effective in decreasing mesenteric vascular resistance. In certain embodiments, wherein the method for treating or preventing a disorder comprises applying a signal to a hypertensive male or normotensive male or female subject's ADN, the method may be particularly effective in decreasing femoral vascular resistance. In certain embodiments, wherein the method for treating or preventing a disorder comprises applying a signal to a normotensive female subject's ADN, the method may elicit a biphasic response in femoral vascular resistance (FVR), for example, the method may elicit an initial decrease in FVR followed by an increase in FVR.

The present disclosure further provides an electrical waveform for use in reversibly modulating (e.g. stimulating) neural activity of a subject's ADN and/or CSN, wherein the waveform is comprised of a plurality of pulse trains of square or sawtooth pulses, the plurality of pulse trains delivered at a frequency of ≤100 Hz, such that when applied to a subject's ADN and/or CSN, the waveform modulates the neural activity of the ADN and/or CSN, wherein the total intensity of the waveform received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In another example, the pulse trains may comprise a series of time periods in which a non-DC (or AC) signal is applied separated by time periods in which a signal is not applied. The non-DC signal may be a pulse, a series of pulses or burst of pulses or the like. The pulse train may apply constant or intermittent stimulation. In certain embodiments, the electrical waveform is for use in reversibly modulating the neural activity of a subject's left ADN.

The present disclosure provides the use of a system for treating a disorder associated with malfunction or loss of the baroreceptor reflex in a subject, for example, in a subject who suffers from or is at risk of suffering a disorder associated with malfunction or loss of the baroreceptor reflex, by applying a signal to the subject's aortic depressor nerve (ADN) and/or carotid sinus nerve (CSN) to reversibly modulate the neural activity of the nerve, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle.

The present disclosure also provides charged particles for use in a method of treating or preventing a disorder associated with malfunction or loss of the baroreceptor reflex, wherein the charged particles cause reversible depolarization of the nerve membrane of the aortic depressor nerve (ADN) and/or carotid sinus nerve (CSN), such that an action potential is generated de novo in the modified nerve, wherein the neural activity of the modified nerve is modulated to produce a change in a physiological parameter in the subject, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received the ADN and/or CSN to produce a ≤30 mmHg drop in the mean arterial blood pressure, and/or wherein the signal is an intermittent signal with a predetermined duty cycle. In certain embodiments, wherein the charged particles reversibly depolarize the nerve membrane of a subject's ADN, the charged particle may be particularly effective in producing a decrease in heart rate. In certain embodiments, wherein the charged particles reversibly depolarize the nerve membrane of a subject's ADN, the charge particles may be particularly effective in decreasing mesenteric vascular resistance. In certain embodiments, wherein the charged particles reversibly depolarize the nerve membrane of a hypertensive male or normotensive male or female subject's ADN, the charged particles may be particularly effective in decreasing femoral vascular resistance. In certain embodiments, wherein the charged particles reversibly depolarize the nerve membrane of a normotensive female subject's ADN, the charged particles may elicit a biphasic response in femoral vascular resistance (FVR), for example, the charged particles may elicit an initial decrease in FVR followed by an increase in FVR.

The present disclosure also provides a modified ADN and/or CSN to which one or more neural interfacing elements of the system of the present disclosure is attached, wherein the one or more neural interfacing element is in signaling contact with the nerve and so the nerve can be distinguished from the nerve in its natural state, and wherein the nerve is located in a patient who suffers from, or is at risk of, a disorder associated with malfunction or loss of the baroreceptor reflex.

The present disclosure also provides a modified ADN and/or CSN, wherein the nerve membrane is reversibly depolarized by charged particles induced by applying an electrical signal, such that an action potential is generated de novo in the modified nerve, wherein the neural activity of the modified nerve is modulated to produce a change in a physiological parameter in the subject, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In certain embodiments, wherein the modified nerve is an ADN, the de novo generation of an action potential may be particularly effective in producing a decrease in heart rate. In certain embodiments, wherein the modified nerve is an ADN, the de novo generation of an action potential may be particularly effective in decreasing mesenteric vascular resistance. In certain embodiments, wherein the modified nerve is an ADN from a hypertensive male or normotensive male or female, the de novo generation of an action potential may be particularly effective in decreasing femoral vascular resistance. In certain embodiments, wherein the modified nerve is an ADN from a normotensive female subject, the de novo generation of an action potential may elicit a biphasic response in femoral vascular resistance (FVR), for example, the action potential may elicit an initial decrease in FVR followed by an increase in FVR.

The present disclosure also provides a modified ADN and/or CSN bounded by a nerve membrane, comprising a distribution of potassium and sodium ions movable across the nerve membrane to alter the electrical membrane potential of the nerve so as to propagate an action potential along the nerve in a normal state; wherein at least a portion of the ADN and/or CSN is subject to the application of a temporary external electrical field which modifies the concentration of potassium and sodium ions within the nerve, causing depolarization of the nerve membrane, thereby, in a disrupted state, temporarily generating an action potential de novo across that portion; wherein the nerve returns to its normal state once the external electrical field is removed, such that the signal reversibly modulates neural activity of the ADN and/or CSN to produce a change in a physiological parameter in the subject, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In certain embodiments, wherein the modified nerve is an ADN, the application of the temporary external electrical field may be particularly effective in producing a decrease in heart rate. In certain embodiments, wherein the modified nerve is an ADN, the application of the temporary external electrical field may be particularly effective in decreasing mesenteric vascular resistance. In certain embodiments, wherein the modified nerve is an ADN from a hypertensive male or normotensive male or female, the application of the temporary external electrical field may be particularly effective in decreasing femoral vascular resistance. In certain embodiments, wherein the modified nerve is an ADN from a normotensive female subject, the application of the temporary external electrical field may elicit a biphasic response in femoral vascular resistance (FVR), for example, the temporary external electrical field may elicit an initial decrease in FVR followed by an increase in FVR.

The present disclosure also provides a modified ADN and/or CSN obtainable by modulating neural activity of the ADN and/or CSN according to a method of the present disclosure.

The present disclosure also provides a method of modifying the neural activity of a subject's ADN and/or CSN, comprising a step of applying a signal to the nerve in order to reversibly modulate (e.g. stimulate) the neural activity of the nerve in a subject, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In a particular aspect, the method does not involve a method for treatment of the human or animal body by surgery. The subject already carries a system of the present disclosure which is in signaling contact with the nerve. In certain embodiments, the method comprises a step of applying a signal to the nerve in order to reversibly modulate the neural activity of the left ADN of a subject.

The present disclosure also provides a method of controlling a system of the present disclosure which is in signaling contact with the ADN and/or CSN, comprising a step of sending control instructions to the system, in response to which the system applies a signal to the ADN and/or CSN.

The present disclosure also provides a computer system implemented method, wherein the method comprises applying a signal to a subject's ADN and/or CSN via at least one neural interfacing element having at least one electrode, such that the signal reversibly modulates the neural activity of the ADN and/or CSN to produce a change in a physiological parameter in the subject, wherein the at least one electrode is suitable for placement on, in, or around the ADN and/or CSN, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In certain embodiments, wherein the computer system implemented method comprises applying a signal to a subject's ADN, the computer system implemented method may be particularly effective in producing a decrease in heart rate. In certain embodiments, wherein the computer system implemented method comprises applying a signal to a subject's ADN, the computer system implemented method may be particularly effective in decreasing mesenteric vascular resistance.

In certain embodiments, wherein the computer system implemented method comprises applying a signal to an ADN from a hypertensive male or normotensive male or female subject, the computer system implemented method may be particularly effective in decreasing femoral vascular resistance. In certain embodiments, wherein the computer system implemented method comprises applying a signal to an ADN from a normotensive female subject, the computer system implemented method may elicit a biphasic response in femoral vascular resistance (FVR), for example, computer system implemented method may elicit an initial decrease in FVR followed by an increase in FVR.

A computer comprising a processor and a non-transitory computer readable storage medium carrying an executable computer program comprising code portions which when loaded and run on the processor cause the processor to: apply a signal to a subject's ADN and/or CSN via at least one neural interfacing element having at least one electrode, such that the signal reversibly modulates the neural activity of the ADN and/or CSN to produce a change in a physiological parameter in the subject, wherein the at least one electrode is suitable for placement on, in, or around the ADN and/or CSN, wherein the change in the physiological parameter is one or more of the group consisting of: a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception, wherein the total intensity of the signal received by the nerve is below a predetermined threshold, the predetermined threshold defined as the total intensity of a signal required to be received by the ADN and/or CSN to produce a ≤30 mmHg drop in the mean arterial blood pressure, and/or wherein the signal is an intermittent signal with a predetermined duty cycle. In certain embodiments, wherein the computer causes the processor to apply a signal to a subject's ADN, the signal may be particularly effective in producing a decrease in heart rate. In certain embodiments, wherein the computer causes the processor to apply a signal to a subject's ADN, the signal may be particularly effective in decreasing mesenteric vascular resistance. In certain embodiments, wherein the computer causes the processor to apply a signal to an ADN from a hypertensive male or normotensive male or female subject, the signal may be particularly effective in decreasing femoral vascular resistance. In certain embodiments, wherein the computer causes the processor to apply a signal to an ADN from a normotensive female subject, the signal may elicit a biphasic response in femoral vascular resistance (FVR), for example, the signal may elicit an initial decrease in FVR followed by an increase in FVR.

### DETAILED DESCRIPTION

Aortic depressor nerve (ADN) and carotid sinus nerve (CSN) The majority of baroreceptor afferent fibers (also called baroafferent fibers) emanate from the aortic arch and both carotid sinuses (see FIG. 1).

The left and right aortic depressor nerves (ADNs) carry baroreceptor afferent fibers that emanate from the aortic arch [25,26,27,28,29]. The ADNs merge with the superior laryngeal nerve, and their cell bodies are located within the inferior vagal (nodose) ganglia in the vagus nerve [1-8]. The left and right carotid sinus nerves (CSNs) carry baroreceptor afferent fibers that emanate from the ipsilateral carotid sinus and chemoafferent fibers from the ipsilateral carotid body [25-29]. The CSNs merge with the glossopharyngeal nerves, and their cell bodies are located within the petrosal ganglia [1-8]. The baroreceptor afferents terminate within their ipsilateral nucleus tractus solitarius (NTS) in the dorsal medulla oblongata. There are distinct differences with respect to the precise termination sites within the subnuclei of the NTS [25-29] and the projections of their first-order NTS neurons (those receiving afferent inputs) to other nuclei within the medulla oblongata and pons and to nuclei above the level of the pons including the hypothalamus and amygdala. [25-29,30,31].

Thus, the ADN and the CSN naturally project baroreceptor activities to the brain. Electrical modulation of the baroreceptor afferent fibers in the ADN and/or CSN bypasses the baroreceptor mechano-sensory transduction and provides data about the central processing of the afferent input and the properties of central and efferent components of the baroreceptor reflex. Electrical modulation allows for precise control of afferent signals transmitted to the nucleus of the tractus solitarius. Hence, by modulating (e.g. stimulating) neural activity of the ADN and/or CSN, it is possible to modulate the baroreceptor reflex, resulting in restoration of the body's homeostatic mechanisms, such as the cardiovascular system (e.g. maintaining blood pressure at nearly constant levels) and the pain regulatory system in various disorders, such as cardiovascular disorders and disorders associated therewith (e.g. pain).

The present disclosure can apply an electrical signal to modulate (e.g. stimulate) neural activity at any point along the ADN. In one aspect, the signal application site is at the cranial portion of the nerve, e.g. below its juncture with the superior laryngeal nerve. This region of the ADN may be more distinct and hence more amenable to electrode attachment compared to the caudal portion where it branches and forms a plexus. An example of signal application site is at position (1) in FIG. 1. In certain embodiments, the present disclosure can apply an electrical signal to modulate (e.g. stimulate) neural activity at any point along the left ADN.

The present disclosure can apply an electrical signal to modulate (e.g. stimulate) neural activity at any point along the CSN. In one aspect, the signal application site is at the cranial portion of the nerve, e.g. below its junction with the glossopharyngeal nerve. This region of the CSN is more distinct and hence more amenable to electrode attachment compared to the caudal portion where it branches and forms a plexus. An example of signal application site is at position (2) in FIG. 1.

The correct identification of the ADN and/or CSN can be confirmed by observing its typical pattern of discharge synchronous with arterial pulsepressure.

Each individual mammalian subject has a left and a right ADN, and a left and a right CSN. The present disclosure may apply a signal to modulate (e.g. stimulate) the ADN and/or CSN unilaterally or bilaterally.

The present disclosure may involve modulating (e.g. stimulating) the ADN.

The present disclosure may involve modulating (e.g. stimulating) the CSN.

The present disclosure may involve modulating (e.g. stimulating) both the ADN and the CSN.

Hence, the present disclosure may involve modulating (e.g. stimulating) the ADN and/or CSN in the following ways:
(1) ADN unilaterally;
(2) CSN unilaterally;
(3) ADN bilaterally;
(4) CSN bilaterally;
(5) ADN unilaterally and CSN unilaterally;
(6) ADN unilaterally and CSN bilaterally;
(7) ADN bilaterally and CSN unilaterally;
(8) ADN bilaterally and CSN bilaterally;
(9) Left ADN unilaterally;
(10) Left ADN unilaterally and CSN unilaterally; or
(11) Left ADN unilaterally and CSN bilaterally.

In aspects of the present disclosure involving unilateral modulation (e.g. stimulation) of the neural activity of the ADN and/or CSN (i.e. options (1)-(2), (5)-(8), (9)-(11) above), the left or the right nerve may be modulated. In certain embodiments of the present disclosure involving unilateral modulation (e.g. stimulation), the left ADN may be modulated.

In aspects of the present disclosure involving unilateral modulation (e.g. stimulation) of the neural activity of both the ADN and CSN (i.e. option (5) or (10) above), the signals are applied to modulate (e.g. stimulate) the nerves ipsilaterally.

In aspects of the present disclosure involving modulating (e.g. stimulating) the neural activity more than one nerve (i.e. options (3)-(8) and (10)-(11) above), the signals may be applied simultaneously or sequentially. In one aspect of the disclosure, the signals are applied simultaneously.

Modulation of Neural Activity

The present disclosure involves modulation of the neural activity of the ADN and/or the CSN. As used herein, “neural activity” of a nerve means the signaling activity of the nerve, for example the amplitude, frequency and/or pattern of action potentials in the nerve. The term “pattern”, as used herein in the context of action potentials in the nerve, is intended to include one or more of: local field potential(s), compound action potential(s), aggregate action potential(s), and also magnitudes, frequencies, areas under the curve and other patterns of action potentials in the nerve or sub-groups (e.g. fascicules) of neurons therein.

Modulation of neural activity, as used herein, is taken to mean that the signaling activity of the nerve is altered from the baseline neural activity—that is, the signaling activity of the nerve in the subject prior to any intervention. Modulation may involve creation of action potentials in the ADN and/or CSN compared to baseline activity. The modulation of the ADN and/or CSN according to the present disclosure results in preferential increased sympathetic signals to the brain.

The present disclosure preferentially stimulates the neural activity of the ADN and/or CSN.

Stimulation may result in the neural activity in at least part of the ADN or CSN being increased compared to baseline neural activity in that part of the nerve. This increase in activity can be across the whole nerve, in which case neural activity is increased across the whole nerve. Thus stimulation may apply to both afferent and efferent fibers of the ADN and/or CSN, but in some aspects of the present disclosure modulation may apply only to afferent fibers or only to efferent fibers. In one aspect, the stimulation applies to afferent fibers.

Stimulation typically involves increasing neural activity e.g. generating action potentials beyond the point of the stimulation in at least a part of the ADN and/or CSN. At any point along the axon, a functioning nerve will have a distribution of potassium and sodium ions across the nerve membrane. The distribution at one point along the axon determines the electrical membrane potential of the axon at that point, which in turn influences the distribution of potassium and sodium ions at an adjacent point, which in turn determines the electrical membrane potential of the axon at that point, and so on. This is a nerve operating in its normal state, wherein action potentials propagate from point to adjacent point along the axon, and which can be observed using conventional experimentation.

One way of characterizing a stimulation of neural activity is a distribution of potassium and sodium ions at one or more points in the axon, which is created not by virtue of the electrical membrane potential at adjacent a point or points of the nerve as a result of a propagating action potential, but by virtue of the application of a temporary external electrical field. The temporary external electrical field artificially modifies the distribution of potassium and sodium ions within a point in the nerve, causing depolarization of the nerve membrane that would not otherwise occur. The depolarization of the nerve membrane caused by the temporary external electrical field generates de novo action potential across that point. This is a nerve operating in a disrupted state, which can be observed by a distribution of potassium and sodium ions at a point in the axon (the point which has been stimulated) that has an electrical membrane potential that is not influenced or determined by the electrical membrane potential of an adjacent point.

Stimulation of neural activity is thus understood to be increasing neural activity beyond the point of signal application. Thus, the nerve at the point of signal application is modified in that the nerve membrane is reversibly depolarized by an electric field, such that a de novo action potential is generated and propagates through the modified nerve. Hence, the nerve at the point of signal application is modified in that a de novo action potential is generated.

As discussed herein, the present disclosure uses an electrical signal, and so the stimulation is based on the influence of electrical currents (e.g. charged particles, which may be one or more electrons in an electrode attached to the nerve, or one or more ions outside the nerve or within the nerve, for instance) on the distribution of ions across the nervemembrane.

Stimulation of neural activity encompasses full stimulation of neural activity in the nerve—that is, aspects of the present disclosure where the total neural activity is increased in the whole nerve. Stimulation of neural activity may be partial stimulation. Partial stimulation may be such that the total signaling activity of the whole nerve is partially increased, or that the total signaling activity of a subset of nerve fibers of the nerve is fully increased (i.e. there is no neural activity in that subset of fibers of the nerve), or that the total signaling of a subset of nerve fibers of the nerve is partially increased compared to baseline neural activity in that subset of fibers of the nerve. For example, an increase in neural activity of 5%, 10%, 15%, 20%, 25%, 30%, 35%, 40%, 45%, 50%, 60%, 70%, 80%, 90% or 95%, or an increase of neural activity in a subset of nerve fibers of the nerve. The neural activity may be measured by methods known in the art, for example, by the number of action potentials which propagate through the axon and/or the amplitude of the local field potential reflecting the summed activity of the action potentials.

The present disclosure may selectively stimulate nerve fibers of various sizes within a nerve. Larger nerve fibers tend to have a lower threshold for stimulation than smaller nerve fibers. Thus, for example, increasing signal amplitude (e.g. increasing amplitude of an electric signal) may generate stimulation of the smaller fibers as well as larger fibers. For example, asymmetrical (triangular instead of square pulse) waveforms may be used stimulate C-fibers (unmyelinated).

One advantage of the present disclosure is that modulation of neural activity is reversible. Hence, the modulation of neural activity is not permanent. For example, upon cessation of the application of a signal, neural activity in the nerve returns substantially towards baseline neural activity within 1-60 seconds, or within 1-60 minutes, or within 1-24 hours (e.g. within 1-12 hours, 1-6 hours, 1-4 hours, 1-2 hours), or within 1-7 days (e.g. 1-4 days, 1-2 days). In some instances of reversible modulation, the neural activity returns substantially fully to baseline neural activity. That is, the neural activity following cessation of the application of a signal is substantially the same as the neural activity prior to a signal being applied. Hence, the nerve or the portion of the nerve has regained its normal physiological capacity to propagate action potentials.

In other aspects of the present disclosure, modulation of the neural activity may be substantially persistent. As used herein, “persistent” is taken to mean that the modulated neural activity has a prolonged effect. For example, upon cessation of the application of a signal, neural activity in the nerve remains substantially the same as when the signal was being applied—i.e. the neural activity during and following signal application is substantially the same.

Disorders Associated with the Malfunction or Loss of the Barorece Tor Reflex

The present disclosure is useful in treating and/or preventing disorders by modulating the baroreceptor reflex. The present disclosure involves treating disorders that are associated with the malfunction or loss of the baroreceptor reflex. These disorders include disorders that are associated with impaired baroreceptor reflex sensitivity. Examples of these disorders include cardiovascular disorders and disorders associated therewith, and cardiorespiratory disorders and disorders associated therewith, as explained further below.

Hypertension

The present disclosure is particularly useful for treating and/or preventing hypertension, such as drug-resistant hypertension. Thus, electrical modulation (e.g. stimulation), such as continuous electrical stimulation, of the ADN and/or CSN in a subject is capable of reducing the resting arterial blood pressure in hypertensive subjects, thereby useful in treating and/or preventing hypertension (e.g. drug-resistant hypertension). The subject may have a systolic blood pressure of ≥140 mmHg and a diastolic blood pressure of ≥90 mmHg. It is known that the blood pressure levels of a normal resting subject are: systolic ≤120 mmHg and diastolic ≤80 mmHg.

The inventors surprising found that electrical stimulation of the ADN is capable of eliciting profound reductions in the levels of arterial blood pressure in normotensive and hypertensive subjects (see example below). The inventors also found that intermittent electrical stimulation of the ADN results in a sustained reductions in arterial blood pressure in Spontaneously Hypertensive rats, and the sustained reduction in arterial blood pressure corresponds to an increase in the disposition of functional proteins in the plasma membranes of baroafferent neurons (see examples below). It is therefore postulated that electrical stimulation of the ADN is capable of causing changes in the molecular mechanisms within the baroafferent pathways including the baroafferents neurons themselves, resulting in sustained reductions in arterial blood pressure. Furthermore, electrical modulation (e.g. stimulation) of the ADN and/or CSN is also useful for overcoming resetting of the baroreflex to lower blood pressure. Baroreceptors reset during prolonged exposure to a high level of arterial blood pressure, and this resetting strongly defends the new level of arterial blood pressure [32,33,34]. Continuous electrical stimulation of the ADN and/or CSN is particularly useful for overcoming resetting of the baroreflex to lower blood pressure.

Interestingly, the inventors found that there were strong gender differences in the hemodynamic responses elicited by electrical stimulation of the ADN in male and female rats (see example below). For example, stimulation of the left ADN in females elicits dramatically greater depressor responses than in males. It is postulated that this may be due to the expression of unique proteins in ADNs of female rats [35,36]. Thus, electrical modulation (e.g. stimulation) of the ADN and/or CSN is capable of lowering arterial pressure in hypertensive females, thereby treating and/or preventing hypertension in females, such as drug-resistant hypertension in female humans. In another example, enhanced hypotensive responses to left ADN stimulation in male SHR are likely driven by more potent baroreflex-mediated reductions in HR and FVR relative to females.

The inventors have also found that there were strong geometric differences in the hemodynamic response elicited by electrical stimulation of the ADN in both normotensive and hypertensive male and normotensive female rats (see example below). More specifically, unilateral stimulation of the left ADN in both males and females elicits greater depressor responses than stimulation of the right ADN. Thus, unilateral electrical modulation (e.g. stimulation) of the left ADN may be more capable of lowering arterial pressure in normotensive males and females and hypertensive males than unilateral electrical modulation of the right ADN, thereby treating and/or preventing hypertension in normotensive males and females and hypertensive males, such as drug-resistant hypertension in male and female humans.

The inventors have also found that there were equivalent hemodynamic responses elicited by electrical stimulation of the left or right ADN in hypertensive female rats (see example below). More specifically, stimulation of either the left or right ADN in hypertensive females appear to elicit equivalent hemodynamic responses, at least with respect to decreasing heart rate and mean arterial pressure. Thus, electrical modulation (e.g. stimulation) of either the left or right ADN is capable of lowering arterial pressure in females, thereby treating and/or preventing hypertension in females, such as drug-resistant hypertension in female humans.

Cardiac Arrhythmias

The present disclosure is particularly useful for treating and/or preventing cardiac arrhythmia, also called cardiac dysrhythmia (or simply irregular heart beat), which refers to a group of conditions in which there is abnormal electrical activity in the heart. Some arrhythmias are life-threatening medical emergencies that can result in cardiac arrest and sudden death. Other cause symptoms such as an abnormal awareness of heart beat. Others may not be associated with any symptoms at all but predispose toward potentially life-threatening stroke, embolus or cardiac arrest. Cardiac arrhythmia can be classified by rate (physiological, tachycardia or bradycardia), mechanism (automaticity, re-entry or fibrillation) or by site of origin (ventricular or supraventricular).

It has been established that low-level carotid baroreceptor stimulation suppresses ventricular arrhythmias during acute ischemia in anesthetized dogs. [37,38] The inventors found that electrical stimulation of the ADN eliminated ventricular arrhythmias in Sprague-Dawley rats with induced congestive heart failure (coronary occlusion model) (see example below).

Thus, electrical modulation (e.g. stimulation) of the ADN and/or CSN is capable of reducing ventricular arrhythmias in hypertensive subjects, thereby useful in treating and/or preventing cardiac arrhythmia.

Diastolic Dysfunction

The present disclosure is also useful in treating cardiac diastolic dysfunction. Autonomic dysfunction accompanied by impaired baroreflex sensitivity is associated with much higher mortality in humans. For example, in rats, baroreflex dysfunction is associated with cardiac diastolic dysfunction independently of the presence of other risk factors [39].

Thus, electrical modulation (e.g. stimulation) of the ADN and/or CSN, such as low-level electrical stimulation (e.g. the total intensity of the signal received by the nerve is below a predetermined threshold as described herein), is capable of treating and/or preventing cardiac diastolic dysfunction, particularly in humans with impaired baroreflex sensitivity.

Myocardial Ischemia

The present disclosure is also useful in treating and/or preventing myocardial ischemia. Thus, electrical modulation (e.g. stimulation) of the ADN and/or CSN is capable of treating and/or preventing myocardial ischemia, such as myocardial ischemia-reperfusion injury. Low-level carotid baroreceptor stimulation (LL-CBS) has been reported to attenuate myocardial ischemia-reperfusion injury and tested underlying molecular mechanisms in adult dogs [40]. This cardioprotective effect of LL-CBS was due inhibition of inflammation, oxidative stress, and apoptosis and modulating Cx43 expression.

Vascular Dementias

The present disclosure is also useful in treating and/or preventing vascular dementias and disorders associated with vascular dementias, such as Alzheimer's disease. Adequate cerebral blood flow perfusion of the brain at rest and under conditions of enhanced circuit activity is essential to maintaining the health of neurons and glial cells [41,42,43]. Reduced cerebral blood flow (hypo-perfusion) directly causes dementias that are collectively known as vascular dementias and plays a vital role in the etiology and maintenance of other dementias such as Alzheimer's disease [41-43]. The diminished blood flow and poor autoregulatory behavior is due to inadequate blood supply and not reduced metabolic demand [41-43]. A functional baroreceptor reflex is essential to maintaining cerebral blood flow and impaired baroreceptor reflex function is directly responsible for cerebral hypoperfusion [44,45,46,47,48,49,50,51]. It has been established that electrical stimulation of the ADN can increase cerebral blood flow in rabbits [52]. Moreover, the inventors found that low-intensity electrical stimulation of the ADN elicits profound increases in blood flow within the brainstem and cortex of anesthetized Sprague-Dawley rats at stimulus intensities that minimally affect systemic arterial blood pressures and other hemodynamic variables (see examples below).

Thus, electrical modulation (e.g. stimulation) of the ADN and/or CSN, e.g. electrical stimulation at low intensity (e.g. the total intensity of the signal received by the nerve is below a predetermined threshold as described herein), is capable of increasing the blood flow within the brainstem and cortex, thereby treating and/or preventing vascular dementia and disorders associated with vascular dementia, such as Alzheimer's disease.

Disorders Associated with Hemodynamic Changes During Sleep and Arousal

The present disclosure is also useful in treating and/or preventing disorders associated with hemodynamic changes during sleep and arousal. The ADN and the CSN play a fundamental role in buffering the changes in hemodynamic variables during sleep and arousal [53,54,55,56,57,58,59,60].

Impairment of baroafferent function results in dramatically augmented responses that are life-threatening.

Thus, electrical modulation (e.g. stimulation) of the ADN and/or CSN is capable of limiting expression of exaggerated hemodynamic responses, thereby treating and/or preventing disorders associated with hemodynamic changes during sleep and arousal, such as cardiorespiratory disorders during sleep (e.g. sleep apnea) and sudden infant death syndrome.

Acute Blood Pressure Changes During Sleep and Arousal

Electrical modulation (e.g. stimulation) of the ADN and/or CSN is also useful for treating and/or preventing acute blood pressure changes in a subject having compromised baroreceptor reflex function and/or compromised cardiovascular system function.

For example, the acute blood pressure changes may be during sleep and arousal. Many animals, including humans, naturally have short-term blood pressure variations throughout the day [61,62].

In some aspects of the present disclosure, the signal is applied prior to waking.

Hyperalgesia

The present disclosure is also useful as an analgesic. For example, the present disclosure is particularly useful for treating hyperalgesia, such as hypertension-associated hyperalgesia. It has been reported that high energy electrical stimulation of the ADN elicited profound analgesic responses [63,64] and the loss of ADN input to the brain resulted in exaggerated nociceptive vagal afferent vagal input [65]. Typically, patients use opioids for pain relief, but the chronic use of opioids are fraught with difficulties for the patient and risks such as addiction and the body's becoming used to the drug (tolerance) can occur. The present disclosure is an improvement from the chronic use of opioids because these risks are minimized.

Thus, electrical modulation (e.g. stimulation) of the ADN and/or CSN, e.g. e.g. electrical stimulation at low intensity (e.g. the total intensity of the signal received by the nerve is below a predetermined threshold described as herein), is capable for the treatment of hyperalgesia, e.g. hypertension-associated hyperalgesia.

Therapy Assessment

Treatment of the disorders described above can be assessed in various ways, but typically involves determining an improvement in one or more physiological parameters of the subject. As used herein, an “improvement in a response” is taken to mean that, for any given response in a subject, an improvement is a change in a value indicative of that response (i.e. a change in a physiological parameter) in the subject towards the normal value or normal range for that value—i.e. towards the expected value in a healthy subject.

As used herein, worsening of cardiac function is taken to mean that, for any given response in a subject, worsening is a change in a value indicative of that response in the subject away from the normal value or normal range for that value—i.e. away from the expected value in a healthy subject.

The present disclosure may also involve detecting one or more physiological parameters of the subject indicative of cardiac function. This may be done before, during and/or after modulation of neural activity in the ADN and/or CSN. The physiological parameter may be organ-based or neuro-based.

Thus, in certain aspects of the present disclosure, the present disclosure further comprises a step of determining one or more physiological parameters of the subject, wherein the signal is applied only when the determined physiological parameter meets or exceeds a predefined threshold value. In such aspects of the present disclosure wherein more than one physiological parameter of the subject is determined, the signal may be applied when any one of the determined physiological parameters meets or exceeds its threshold value, alternatively only when all of the determined physiological parameters meet or exceed their threshold values. In certain aspects of the present disclosure wherein the signal is applied by a system of the present disclosure, the system further comprises at least one detector configured to determine the one or more physiological parameters of the subject.

In certain aspects of the present disclosure, the physiological parameter is an action potential or pattern of action potentials in a nerve of the subject, wherein the action potential or pattern of action potentials is associated with the condition that is to be treated.

An organ-based biomarker may be any measurable physiological parameter of the heart, the circuitry system, the respiratory system, the brain or the sensory system. For example, a physiological parameter may be one or more of the group consisting of: systemic arterial blood pressure (systolic pressure, diastolic pressure, or mean arterial pressure), heart rate, heart rhythm, electrical conduction in the heart and heart contractility (e.g. ventricular pressure, ventricular contractility, activation-recovery interval, effective refractory period, stroke volume, ejection fraction, end diastolic fraction, stroke work, arterial elastance), vascular resistance (e.g. total peripheral resistance, mesenteric vascular resistance or femoral vascular resistance), cardiac output, rate of blood flow (e.g. systemic blood flow, or cerebral blood flow), minute ventilation, and pain perception. The physiological parameters related to heart and the circuitry system may indicate a hemodynamic response, chronotropic response, a dromotropic response, a lusitropic response and/or an inotropic response.

Blood pressure can be monitored either invasively through an inserted blood pressure transducer assembly (providing continuous monitoring), or noninvasively by repeatedly measuring the blood pressure with an inflatable blood pressure cuff, e.g. a sphygmomanometer. For example, the blood pressure levels of a normal resting subject are: systolic ≤120 mmHg and diastolic ≤80 mmHg. A subject having hypertension typically have a systolic blood pressure of ≥140 mmHg and a diastolic blood pressure of ≥90 mmHg.

The present disclosure may involve assessing the heart rate by methods known in the art, for example, with a stethoscope or by feeling peripheral pulses. These methods cannot usually diagnose specific arrhythmias but can give a general indication of the heart rate and whether it is regular or irregular.

Not all of the electrical impulses of the heart produce audible or palpable beats; in many cardiac arrhythmias, the premature or abnormal beats do not produce an effective pumping action and are experienced as “skipped” beats.

Heart Rate Variability (HRV) a technique useful for assess autonomic balance. HRV relates to the regulation of the sinoatrial node, the natural pacemaker of the heart by the sympathetic and parasympathetic branches of the autonomic nervous system. An HRV assessment is based on the assumption that the beat-to-beat fluctuations in the rhythm of the heart provide us with an indirect measure of heart health, as defined by the degree of balance in sympathetic and parasympathetic nerve activity.

The present disclosure may also involve assessing the heart rhythm. For example, the simplest specific diagnostic test for assessment of heart rhythm is the electrocardiogram (abbreviated ECG or EKG). A Holter monitor is an EKG recorded over a 24-hour period, to detect arrhythmias that can happen briefly and unpredictably throughout the day.

Other useful assessment techniques include using a cardiac event recorder, an electrophysiological (EP) study, an echocardiogram, a nuclear scan, a coronary angiography, a cardiac CT scan, a stress test, a brain CT scan for signs of stroke, MRI scan for providing detailed information about the blood vessel damage.

Vascular resistance (for example, total peripheral resistance, mesenteric vascular resistance or femoral vascular resistance) can be derived from the change in blood pressure across the circulation loop and the blood flow (e.g. cardiac output).

The present disclosure may also involve measuring the level of brain natriuretic peptide or B-type natriuretic peptide (BNP) (also called ventricular natriuretic peptide or natriuretic peptide B), which is a biomarker for diagnosing heart failure. BNP is secreted by the ventricles of the heart in response to excessive stretching of cardiomyocytes.

Respiration parameters may also be useful. They can be derived from, for example, a minute ventilation signal and a fluid index can be derived from transthoracic impedance. For example decreasing thoracic impedance reflects increased fluid buildup in lungs, and indicates a progression of heart failure.

Respiration can significantly vary minute ventilation. The transthoracic impedance can be totaled or averaged to provide an indication of fluid buildup.

For vascular dementias, mental abilities are often assessed, e.g. the mini mental state examination (MMSE).

The present disclosure may involve assessing a neuro-based biomarker. Hence, in some aspects of the present disclosure, the physiological parameter may be one or more abnormal cardiac electrical signals from the subject indicative of cardiac dysfunction. The abnormal cardiac electrical signals may be measured in a cardiac-related intrathoracic nerve or peripheral ganglia of the cardiac nervous system. The abnormal electric signals may be a measurement of cardiac electric activity.

Example of assessing cardiac electrical signals includes microneurography or plasma noradrenaline concentration. Microneurography involves using fine electrodes to record ‘bursts’ of activity from multiple or single afferent and efferent nerve axons [66,67]. The measurement of regional plasma noradrenaline spillover is useful in providing information on sympathetic activity in individual organs. Following nerve depolarization, any remaining noradrenaline in the synapse, the ‘spillover’, is washed out into the plasma and the plasma concentration is therefore directly related to the rate of sympathetic neuronal discharge [68,69,70].

For example, in a subject having or is at risk of a cardiovascular disorder, an improvement in a physiological parameter or in a response of the subject may be indicated by, a decrease in mean arterial pressure, a decrease in heart rate, an increase in minute ventilation, an improvement in the regularity of the heart rhythm, an improvement in heart conduction, an increase in heart contractility, a decrease in vascular resistance (e.g. total peripheral resistance, mesenteric vascular resistance or femoral vascular resistance), an increase in cardiac output, an increase in blood flow (e.g. systemic blood flow, or cerebral blood flow), an increase in minute ventilation, an increase in a hemodynamic response, a decrease in a chronotropic evoked response, a decrease in a dromotropic evoked response, a decrease in a lusitropic evoked response, a decrease in an inotropic evoked response. In another example, an improvement in a physiological parameter or in a response of the subject, in particular in a normotensive female subject, may be indicated by a biphasic response in femoral vascular resistance.

For example, in a subject having or is at risk of hyperalgesia (e.g. hypertensive-associated hyperalgesia), an improvement in a physiological parameter of the subject may be indicated by a decrease in pain perception. For example, a decrease in the pain number scale, 0 being no pain and being the worst pain imaginable.

In certain aspects of the present disclosure of the present disclosure, treatment and/or prevention of the disorder is indicated by an improvement in the profile of neural activity in the ADN and/or CSN. That is, treatment and/or prevention of the disorder is indicated by the neural activity in the ADN and/or CSN approaching the neural activity in a healthy subject.

As used herein, a physiological parameter is not affected by the modulation of the ADN and/or CSN if the parameter does not change (in response to ADN and/or CSN modulation) from the normal value or normal range for that value of that parameter exhibited by the subject or subject when no intervention has been performed i.e. it does not depart from the baseline value for that parameter.

The skilled person will appreciate that the baseline for any neural activity or physiological parameter in an subject need not be a fixed or specific value, but rather can fluctuate within a normal range or may be an average value with associated error and confidence intervals. Suitable methods for determining baseline values are well known to the skilled person.

As used herein, a physiological parameter is determined in a subject when the value for that parameter exhibited by the subject at the time of detection is determined. A detector (e.g. a physiological sensor subsystem, a physiological data processing module, a physiological sensor, etc.) is any element able to make such a determination.

It will be appreciated that any two physiological parameters may be determined in parallel aspects of the present disclosure, the controller is coupled detect the pattern of action potentials tolerance in the subject.

A predefined threshold value for a physiological parameter is the minimum (or maximum) value for that parameter that must be exhibited by a subject or subject before the specified intervention is applied. For any given parameter, the threshold value may be defined as a value indicative of a pathological state or a disease state. The threshold value may be defined as a value indicative of the onset of a pathological state or a disease state. Thus, depending on the predefined threshold value, the present disclosure can be used as a treatment. Alternatively, the threshold value may be defined as a value indicative of a physiological state of the subject (that the subject is, for example, asleep, post-prandial, or exercising). Appropriate values for any given physiological parameter would be simply determined by the skilled person (for example, with reference to medical standards of practice).

Such a threshold value for a given physiological parameter is exceeded if the value exhibited by the subject is beyond the threshold value—that is, the exhibited value is a greater departure from the normal or healthy value for that physiological parameter than the predefined threshold value.

As explained above, the present disclosure is useful for the prevention of the disorders described above. For example, the present disclosure exerts cardioprotective effects. Hence, subjects who are at risk of developing cardiovascular disorders may be subjected to application of the signals described herein, e.g. resulting in a decrease the arrhythmic burden. The cardiac testing strategies for subjects at risk of cardiac dysfunction are known in the art, e.g. heart rate variability (HRV), baroreflex sensitivity (BRS), heart rate turbulence (HRT), heart rate deceleration capacity (HRDC) and T wave alternans (TWA). Deviation of these parameters from the baseline value range would be an indication of the subject being at risk of developing cardiovascular disorders.

Other indications include when the subject has a history of cardiac problems or a history of myocardium injury. For example, the subject has undergone heart procedures, e.g. heart surgery. The subject may have had a myocardial infarction. The subject may have emphysema or chronic obstructive pulmonary disease. The subject may have a history of arrhythmia or be genetically pre-disposed to arrhythmia. The subject may have diabetes. The subject may have a blood pressure that is higher than normal, such as a systolic blood pressure level of 120-139 mmHg, and a diastolic blood pressure level of 80-89 mmHg. The subject may be genetically pre-disposed to high blood pressure.

The present disclosure may be useful in a subject who has compromised baroreceptor reflex function and/or compromised cardiovascular system function.

The present disclosure may be useful in a subject who suffers from or is at risk of suffering a disorder associated with malfunction or loss of the baroreceptor reflex.

For preventive use, a subject at risk of developing cardiovascular disorders may be subjected to signal application for x min at regular intervals, wherein x=≤3 min, ≤5 min, ≤10 min, ≤20 min, ≤30 min, ≤40 min, ≤50 min, ≤60 min, ≤70 min, ≤80 min, ≤90 min, ≤120 min, or ≤240 min. The interval may be once every day, once every 2 days, once every 3 days etc. The interval may be more than once a day, e.g. twice a day, three times a day etc.

As discussed herein, the system of the present disclosure may comprise a system or device to be implanted into the subject. A subject of the present disclosure may, in addition to having a system of the present disclosure, receive medicine for their condition. For instance, a subject having an implant according to the present disclosure may receive an anti-inflammatory medicine (which will usually continue medication which was occurring before receiving the implant). Such medicines include, nonsteroidal anti-inflammatory drugs (NSAIDs), steroids, 5ASAs, immunosuppressants such as azathioprine, methotrexate and ciclosporin, and biological drugs like infliximab and adalimumab. Thus the present disclosure provides the use of these medicines in combination with a system of the present disclosure.

A subject suitable for the present disclosure may be any age, but will usually be at least 40, 45, 50, 55, 60, 65, 70, 75, 80 or 85 years of age.

A subject suitable for the present disclosure may be males or females. In a particular aspect, the subject is a female.

A System for Implementing the Present Disclosure

A system 116 according to the present disclosure comprises a device, which may be implantable (e.g. implantable device 106 of FIG. 2). The system 116 comprises an electrode 108, comprising exposed portions 109, suitable for placement on or around the ADN and/or CSN surrounding a left gastro epiploic artery or a short gastric artery. The device 106 may also comprises a processor (e.g. microprocessor 113) coupled to the at least one neural interfacing element.

The electrode 108, may take many forms, and includes any component which, when used in an implantable device for implementing the present disclosure, is capable of applying a stimulus or other signal that modulates electrical activity, e.g., action potentials, in a nerve.

The various components of the system may be part of a single physical device, either sharing a common housing or being a physically separated collection of interconnected components connected by electrical leads (e.g. leads 107). As an alternative, however, the present disclosure may use a system in which the components are physically separate, and communicate wirelessly. Thus, for instance, the electrode 108, and the implantable device 106 can be part of a unitary device, or together may form a system 116. In both cases, further components may also be present to form a larger device (e.g. system 100).

Electrical Signal

The present disclosure involves applying a signal via one or more neural interfacing elements (e.g. neural interfacing element 108 in FIG. 2) placed in signaling contact with the ADN and/or CSN. The signal is an electrical signal, which may be, for example, a voltage or current signal. The at least one neural interfacing element of the system (e.g. system 116) is configured to apply the electrical signals to a nerve, or a part thereof. The skilled person will appreciate that electrical signals are just one way of implementing the present disclosure. For example, the signal can be any signal that induces a change in electric field in the area surrounding a portion of the nerve. Notably, the signal can be applied such that the total intensity of the signal received by the nerve is below a predetermined threshold as described herein.

According to FIG. 2, the system 116 may comprise an implantable device 106 which may comprise a signal generator 117. The signal generator 117 is a voltage source or a current source, configured to deliver a voltage signal or a current signal respectively.

Signals applied according to the present disclosure are ideally non-destructive. As used herein, a “non-destructive signal” is a signal that, when applied, does not irreversibly damage the underlying neural signal conduction ability of the nerve. That is, application of a non-destructive signal maintains the ability of the nerve or fibers thereof, or other nerve tissue to which the signal is applied, to conduct action potentials when application of the signal ceases, even if that conduction is in practice artificially stimulated as a result of application of the non-destructive signal.

Total Intensity of a Signal and the Threshold (T_{INT})

The total intensity of a signal received by the nerve refers to the magnitude of the total signal intensity received by the nerve for the duration that the signal is applied, and this is below a predetermined threshold. The total intensity of a signal received by the nerve is defined by amplitude*frequency*pulse width*duration of signal applied. In other words, the total intensity can be determined by the area under the curve of a graphical plot of the electrical signal with amplitude in the y axis and time in the x axis.

The predetermined threshold is defined as the total intensity of a signal required to be received by the ADN and/or CSN to produce a ≤30 mmHg drop in the mean arterial blood pressure. For example, the drop in mean arterial blood pressure may be ≤25 mmHg, ≤20 mmHg, ≤15 mmHg, or ≤10 mmHg.

In some aspects of the present disclosure, the predetermined threshold is defined as the total intensity of a signal required to be received by the ADN and/or CSN to produce a drop in the mean arterial blood pressure of between 30 mmHg and 10 mmHg.

The predetermined threshold may vary according to the subject to which the signal is applied. The threshold may vary by one or more of: age, sex, general health of the user. Thus, the predetermined threshold may be a value that is determined in the subject who will be receiving a signal to modulate the neural activity of the ADN and/or the CSN as described herein, and so the predetermined threshold would be specific to the subject.

Alternatively, the predetermined threshold may be a fixed value. For example, the predetermined threshold may be an average that has been determined across a group of subjects. The group of subjects may be age-specific, gender-specific, and/or disorder-specific. For example, subjects who suffer from or are at risk of a particular disorder associated with malfunction or loss of baroreceptor reflex, as described herein e.g. subjects having hypertension or female subjects having hypertension.

It would be of course understood in the art that the signal received by the nerve would be within clinical safety margins (e.g. suitable for maintaining nerve signaling function, suitable for maintaining nerve integrity, and suitable for maintaining the safety of the subject). The electrical parameters within the clinical safety margin would typically be determined by pre-clinical studies. For example, the frequency of the signal is not higher than 200 Hz, 150 Hz, or 100 Hz. For example, the amplitude of the signal is not larger than 3 mA, 2 mA, or 1 mA.

For example, the predetermined threshold may be determined by applying signals to the ADN and/or CSN with increasing amplitude (mA) at small intervals (e.g. increments of 0.2 mA), each for a constant duration (e.g. 20 s) at a constant frequency (e.g. 5 Hz) and a constant pulse width (e.g. 0.5 ms), and identifying the minimum amplitude (e.g. 0.6 mA) at which a 30 mmHg drop in the mean arterial blood pressure in the subject is produced. Thus, the total intensity of the signal that produces a 30 mmHg drop in the mean arterial blood pressure in the subject is 30 μAs, and so the predetermined threshold is 30 μAs.

By way of a further example, the predetermined threshold may be determined by applying signals to the ADN and/or CSN with increasing frequency (Hz) at small intervals (e.g. increments of 2.5 Hz), each for a constant duration (e.g. 20 s) at a constant amplitude (e.g. 0.6 mA) and a constant pulse width (e.g. 0.5 ms), and identifying the minimum frequency (e.g. 5 Hz) at which a 30 mmHg drop in the mean arterial blood pressure in the subject is produced. Thus, the total intensity of the signal that produces a 30 mmHg drop in the mean arterial blood pressure in the subject is 30 μAs, and so the predetermined threshold is 30 μAs.

By way of a further example, the predetermined threshold may be determined by applying signals to the ADN and/or CSN with increasing the pulse width (ms) at small intervals (e.g. increments of 0.1 ms), each for a constant duration (e.g. 20 s) at a constant amplitude (e.g. 0.6 mA) and a constant frequency (e.g. 5 Hz), and identifying the minimum pulse width (e.g. 0.5 ms) at which a 30 mmHg drop in the mean arterial blood pressure in the subject is produced. Thus, the total intensity of the signal that produces a 30 mmHg drop in the mean arterial blood pressure in the subject is 30 μAs, and so the predetermined threshold is 30 μAs.

By way of a further example, the predetermined threshold may be determined by applying signals to the ADN and/or CSN with increasing the duration (s) of signal application at small intervals (e.g. increments of 5 s), each for a constant pulse width (e.g. 0.5 ms) at a constant amplitude (e.g. 0.6 mA) and a constant frequency (e.g. 5 Hz), and identifying the minimum duration (e.g. 20 s) at which a ≤30 mmHg drop in the mean arterial blood pressure in the subject is produced. Thus, the total intensity of the signal that produces a 30 mmHg drop in the mean arterial blood pressure in the subject is 30 μAs, and so the predetermined threshold is 30 μAs.

In some aspects of the present disclosure, the predetermined threshold, may be ≤30 μAs, ≤28 μAs, ≤26 μAs, ≤24 μAs, ≤22 μAs, ≤20 μAs, ≤18 μAs, ≤16 μAs, ≤14 μAs, ≤12 μAs, ≤10 μAs, ≤8 μAs, ≤6 μAs, ≤4 μAs, ≤2 μAs, ≤1 μAs, ≤0.8 μAs, ≤0.6 μAs, ≤0.4 μAs, ≤0.2 μAs, or ≤0.1 μAs.

In some aspects of the present disclosure, the total signal intensity that produces a ≤30 mmHg drop in the mean arterial blood pressure, and hence the predetermined threshold, is ≤30 μAs.

In some aspects of the present disclosure, the total signal intensity that produces a ≤25 mmHg drop in the mean arterial blood pressure, and hence the predetermined threshold, is ≤16 μAs.

In some aspects of the present disclosure, the total signal intensity that produces a ≤20 mmHg drop in the mean arterial blood pressure, and hence the predetermined threshold, is ≤6 μAs.

In some aspects of the present disclosure, the total signal intensity that produces a ≤15 mmHg drop in the mean arterial blood pressure, and hence the predetermined threshold, is ≤4 μAs.

In some aspects of the present disclosure, the total signal intensity that produces a ≤10 mmHg drop in the mean arterial blood pressure, and hence the predetermined threshold, is ≤2 μAs.

In some aspects of the present disclosure, the predetermined threshold may be defined by the combination of: signal intensity and one or more of the following parameters: (a) frequency, (b) amplitude, (c) pulse width, and (d) signal duration. Examples of these parameters can be found in FIG. 21.

By way of an example, the predetermined threshold for a ≤30 mmHg drop in the mean arterial blood pressure may be defined by the combination of: a signal intensity of ≤30 μAs and one or more of the following parameters: (a) a frequency of ≤5 Hz, (b) an amplitude of ≤0.6 mA, (c) a pulse width of ≤0.5 ms, and (d) a signal duration of ≤20 s.

The present disclosure may involve applying a total signal intensity below a predetermined threshold, also referred to herein as “T_{INT}”. In one aspect, the total signal intensity to be received by the nerve may be between 0.1 T_{INT} and 0.9 T_{INT}. In some aspects of the present disclosure, the total signal intensity to be received by the nerve is between one of: 0.2 T_{INT} and 0.8 T_{INT}, 0.3 T_{INT} and 0.7 T_{INT}, and 0.4 T_{INT} and 0.6 T_{INT}. In certain aspects of the present disclosure, the total signal intensity to be received by the nerve is about: ≤0.1 T_{INT}, ≤0.2 T_{INT}, ≤0.3 T_{INT}, ≤0.4 T_{INT}, ≤0.5 T_{INT}, ≤0.6 T_{INT}, ≤0.7 T_{INT}, ≤0.8 T_{INT}, or ≤0.9 T_{INT}.

Signal Parameters for Modulating Neural Activity

In the above examples, the signal generator 117 is configured to deliver an electrical signal for modulating (e.g. stimulating) the ADN and/or CSN. In the present application, the signal generator 117 is configured to apply an electrical signal with certain electrical signal parameters to modulate (e.g. stimulate) neural activity in the ADN and/or CSN. Signal parameters suitable for the present disclosure are described further below.

The electrical signal may be applied intermittently or continuously. The present disclosure does not use an electrical signal that causes inhibition of neural activity of the nerve, e.g. kilohertz frequency alternating current (KHFAC).

Waveform

The electrical signal may be in square or sawtooth waveform. Other pulse waveforms such as sinusoidal, triangular, trapezoidal, quasitrapezodial or complex waveforms may also be used with the present disclosure.

In some aspects of the present disclosure, the waveform is biphasic. The term “biphasic” refers to a signal which delivers to the nerve over time both a positive and negative charge. In certain aspects, the waveform is charge-balanced. In some aspects, the waveform is non charge-balanced.

Pulse Width

The electrical signal may comprise a pulse train, each pulse with a defined pulse width. The range of pulse widths may be from 0.01 ms to 500 ms, e.g. between 0.05 ms to 100 ms, or between 0.1 ms and 1 ms (including, if applicable, both positive and negative phases of the pulse, in the case of a charge-balanced biphasic pulse). The pulses in the pulse trains may be charge-balanced biphasic pulses. The term “charge-balanced” in relation to a pulse train is taken to mean that the positive charge and negative charge applied by the signal over the pulse duration is equal.

For example, the pulse width may be ≤500 ms, ≤450 ms, ≤400 ms, ≤350 ms, ≤300 ms, ≤250 ms, ≤200 ms, ≤150 ms, ≤100 ms, ≤50 ms, ≤510 ms, ≤5 ms, ≤1 ms, ≤0.8 ms, ≤0.6 ms, ≤0.4 ms, ≤0.2 ms, ≤0.1 ms, ≤0.08 ms, ≤0.06 ms, ≤0.04 ms, ≤0.02 ms, or ≤0.01 ms.

In some aspects of the present disclosure, the pulse width is ≤1 ms, e.g. between 0.1 ms and 1 ms.

Frequency

The electrical signal may have a frequency of 1 Hz to 100 Hz, e.g. between 1 Hz and 50 Hz, between 1 Hz and 30 Hz, or between 1 Hz and 20 Hz. For example, the frequency may be ≤200 Hz, ≤150 Hz, ≤100 Hz, ≤90 Hz, ≤80 Hz, ≤70 Hz, ≤60 Hz, ≤50 Hz, ≤40 Hz, ≤30 Hz, ≤20 Hz, ≤10 Hz, 5 Hz, 2 Hz, or 1 Hz.

In some aspects of the present disclosure, the frequency is ≤20 Hz, e.g. 10 Hz, 5 Hz or 1 Hz.

The signal generator 117 may be configured to deliver one or more pulse trains at intervals according to the above-mentioned frequencies. For example, a frequency of 1 to 100 Hz results in a pulse interval between 1 pulse per second and 100 pulses per second, within a given pulse train.

Amplitude

The electrical signal may have an amplitude between 0.1 to 3 mA, e.g. between 0.2 mA and 2.5 mA, or between 0.4 mA and 2 mA. For example, the amplitude may be ≤3 mA, ≤2.5 mA, ≤2 mA, ≤1.8 mA, ≤1.6 mA, ≤1.4 mA, ≤1.2 mA, ≤1 mA, ≤0.8 mA, ≤0.6 mA, ≤0.4 mA, ≤0.2 mA, or ≤0.1 mA.

In some aspects of the present disclosure, the amplitude is ≤2 mA, e.g. between 0.4 mA and 2 mA. For aspects of the present disclosure where the signal is a pulse train, advantages have noted in respect of pulses with lower amplitudes. For example, pulse amplitudes may be ≤2 mA, e.g. between 0.4 mA and 2 mA.

It will be appreciated by the skilled person that the current amplitude of an applied electrical signal necessary to achieve the intended modulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject.

Duty Cycle

The signal may be applied in a (ONy−OFFz)n pattern, where n>1 and y>0, over a period of time. For example, the signal is applied (i.e. “ON”) for a time period “y”, then stopped (i.e. “OFF”) for a time period “z”, and this pattern is repeated for “n” number of times. y and z may independently be ≤10 s, ≤9 s, ≤8 s, ≤7 s, ≤6 s, ≤5 s, ≤4 s, ≤3 s, ≤2 s, ≤1 s, ≤500 ms, 100 ms, 50 ms, ≤10 ms, ≤1 ms, ≤500 μs, ≤100 μs, ≤50 μs, ≤20 μs, or ≤10 μs. n may be ≤50, ≤40, ≤30, ≤20, ≤10, ≤5, ≤4, ≤3, ≤2.

30 In certain aspects of the present disclosure, the signal is intermittent, i.e. the signal is applied in a (ONy−OFFz)n pattern, where n>1, y>0, z>0, and y and z may independently be ≤10 s, ≤9 s, 8 s, ≤7 s, ≤6 s, 5 s, 4 s, 3 s, 2 s, s, 500 ms, 100 ms, 50 ms, 10 ms, 1 ms, 500 μs, ≤100 μs, ≤50 μs, ≤20 μs, or ≤10 μs. n may be ≤50, ≤40, ≤30, ≤20, ≤10, ≤5, 4, ≤3, K 2. In one aspect of the disclosure, y is 5 s and z is 5 s.

In one aspect of the disclosure, y is 5 s and z is 3 s. The duty cycle describes the proportion of “ON” time to the regular interval or period of time.

In an aspect of the disclosure, the signal may have a predetermined duty cycle of ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1%.

In an aspect of the disclosure, the signal has a predetermined duty cycle of ≤65% or ≤50%.

Duration and Timings of Signal Application

In some aspects of the present disclosure, the signal is applied to the ADN and/or CSN as soon as an increase in the mean arterial blood pressure can be detected, e.g. by the system according to the present disclosure.

In some aspects of the present disclosure, the signal is applied at a specific time of the day, e.g. prior to blood pressure surges. In certain aspects of the present disclosure, the signal is applied prior to waking, e.g. ≤0.5 h, ≤1 h, 1.5 h, ≤2 h, ≤2.5 h or ≤3 h before waking.

In some aspects of the present disclosure, the signal is applied when the mean arterial blood pressure increases by ≥5 mmHg, ≥10 mmHg, ≥15 mmHg, ≥20 mmHg, ≥25 mmHg, ≥30 mmHg, ≥35 mmHg, or ≥40 mmHg over a certain period of time, t. In particular aspects, the signal is applied when the mean arterial blood pressure increases by ≤10 mmHg over a certain period of time, t.

Alternatively, the signal is applied when the mean arterial blood pressure increases by x % from the normal value over a certain period of time, t, wherein x is ≥5%, ≥10%, ≥15%, ≥20%, ≥25%, ≥30%, ≥35%, ≥40%, ≥45% or ≥50% over a certain period of time, t. In particular aspects, the signal is applied when the mean arterial blood pressure increases by 10% over a certain period of time, t.

The certain period of time, t, may be ≤30 min, ≤25 min, ≤20 min, ≤15 min, ≤10 min, ≤5 min, ≤2 min, or ≤1 min.

In the aspects of the present disclosure that involves applying the signal in a (ONy−OFFz)n pattern, where n>1, y>0, z>0, the signal is applied for a specific amount of time, e.g. ≤5 s, ≤10 s, ≤15 s, ≤20 s, ≤25 s, ≤30 s, ≤35 s, ≤40 s, ≤45 s, ≤50 s, ≤55 s, ≤1 min, ≤2 min, ≤3 min, ≤4 min, ≤5 min, ≤10 min, ≤15 min, ≤20 min, ≤25 min or ≤30 min. In a particular aspect, the signal is applied in a (ONy−OFFz)n pattern, where n>1, y>0, z>0, for ≤20 s.

In some aspects of the disclosure, a signal is applied in a (ONy−OFFz)n pattern, where n>1, y>0, z>0, and the signal is applied for 1, ≤2, ≤3, ≤4, ≤5, ≤6, ≤7, ≤8, ≤9, ≤10, 11, or ≤12 times aday.

In some aspects of the disclosure, a signal is applied in a (ONy−OFFz)n pattern, where n>1, y>0, z>0, and the signal is applied for ≤30 min at any given time up to 12 times a day. For example, the signal is applied for ≤5 s, ≤10 s, ≤15 s, ≤20 s, ≤25 s, ≤30 s, ≤35 s, ≤40 s, ≤45 s, ≤50 s, ≤55 s, ≤1 min, ≤2 min, ≤3 min, ≤4 min, ≤5 min, ≤5 min, ≤10 min, ≤15 min, ≤20 min, ≤25 min or ≤30 min.

For example, the signal is applied for 1, ≤2, ≤3, ≤4, ≤5, ≤6, ≤7, ≤8, ≤9, ≤10, 11, or ≤12 times a day.

The signal generator 117 may be pre-programmed to deliver one or more signals with signal parameters falling within the ranges described herein. Alternatively, the signal generator 117 may be controllable to adjust one or more of the signal parameters discussed above while ensuring that the total intensity delivered is below the predetermined threshold. Control may be open loop, wherein the operator of the implantable device 106 may configure the signal generator using an external controller (e.g. controller 101), and warnings may be issued to the operator if the total signal intensity received by the nerve is not below the predetermined threshold. Control may alternatively or additionally be closed loop, wherein signal generator modifies the signal parameters in response to one or more responses of the heart. Open loop and closed loop control of signal parameters is further described below.

It will be appreciated by the skilled person that the signal parameters of an applied electrical signal necessary to achieve the intended modulation of the neural activity will depend upon the positioning of the electrode and the associated electrophysiological characteristics (e.g. impedance). It is within the ability of the skilled person to determine the appropriate variations in signal parameters for achieving the intended modulation of the neural activity in a given subject.

Electrodes

As mentioned above, the system comprises at least one neural interfacing element having at least one electrode (e.g. electrode 108). In some aspects of the present disclosure, the at least one electrode is positioned on a neural interface. The neural interface and/or at least one electrode is configured to at least partially circumvent the nerve. In some aspects of the present disclosure, the neural interface and/or at least one electrode is configured to fully circumvent the nerve.

Electrode types suitable for the present disclosure are known in the art. For example, [71] discloses several types of electrode for non-damaging neural tissue modulation. The document discloses cuff electrodes (e.g. spiral cuff, helical cuff or flat interface), and flat interface electrodes, both of which are also suitable for use with the present disclosure. A mesh, a linear rod-shaped lead, paddle-style lead or disc contact electrode (including multi-disc contact electrodes) are also disclosed in [71] and would be suitable for use in the present disclosure. Further electrodes suitable for the present disclosure are patch electrodes, and stent electrodes [72,73]. Some electrodes may be sewn onto the nerve [74].

Also suitable are intrafascicular electrode, glass suction electrode, paddle electrode, bipolar hemi-cuff electrode, bipolar hook electrode, percutaneous cylindrical electrode. Electrodes may be monopolar, bipolar, tripolar, quadripolar or have five or more poles. The electrodes may fabricated from, or be partially or entirely coated with, a high charge capacity material such as platinum black, iridium oxide, titanium nitride, tantalum, poly(elthylenedioxythiophene) and suitable combinations thereof.

The geometry of the neural interface and/or least one neural interfacing element is defined in part by the anatomy of the ADN and/or CSN. For example, the geometry of the neural interface and/or the at least one neural interfacing element may be limited by the length and/or the diameter of the ADN and/or CSN.

The ADN has a diameter of about 500 μm and about 1 mm, and a length of about 1 cm to about 2 cm. In some aspects of the present disclosure, the geometry of the neural interface and/or least one electrode for placement on or around the ADN may have: (a) a diameter of ≥1 mm, ≥950 μm, ≥900 μm, ≥850 μm, ≥800 μm, ≥750 μm, ≥700 μm, ≥650 μm, ≥600 μm, ≥550 μm, or ≥500 μm; and/or (b) a length of ≤2 cm, ≤1.8 cm, ≤1.6 cm, ≤1.4 cm, ≤1.2 cm, or ≤1 cm.

The CSN has a diameter of about 500 μm and about 1 mm, and a length of about 1 cm to about 2 cm [75]. In some aspects of the present disclosure, the geometry of the neural interface and/or least one electrode placement on or around the CSN may have: (a) a diameter of ≥1 mm, ≥950 μm, ≥900 μm, ≥850 μm, ≥800 μm, ≥750 μm, ≥700 μm, 650 μm, ≥600 μm, ≥550 μm, or ≥500 μm; and/or (b) a length of ≤2 cm, ≤1.8 cm, ≤1.6 cm, ≤1.4 cm, ≤1.2 cm, or ≤1 cm.

The electrodes may be insulated by a non-conductive biocompatible material, which may be spaced transversely along the neural interface and, in use, along the nerve. Typically, the electrode applies the electrical signal by exerting an electrical field across the nerve bundle, and hence applying the electrical signal to many nerve fibers within the bundle. This creates multiple action potentials in each nerve fiber, and the combination of these action potentials may be called a compound action potential.

In some aspects of the present disclosure (for example, FIG. 2), the one or more electrodes may be coupled to implantable device 106 of system 116 via electrical leads 107. Alternatively, implantable device 106 may be directly integrated with the electrodes 108 without leads. In any case, implantable device 106 may comprise DC current blocking output circuits, optionally based on capacitors and/or inductors, on all output channels (e.g. outputs to the electrodes 108, or physiological sensor 111).

The present disclosure may refer to one or more electrode that attaches to the ADN. The present disclosure may refer to one or more electrode that attaches to the CSN.

The present disclosure may refer to one or more electrode that attaches to the ADN and one or more electrode that attaches to the CSN, to modulate (e.g. stimulate) the neural activity of either or both nerves.

The electrodes may attach unilaterally or bilaterally to the ADN and/or CSN.

Hence, the at least one electrode may attach to the ADN and/or CSN in the following ways:
(1) ADN unilaterally;
(2) CSN unilaterally;
(3) ADN bilaterally;
(4) CSN bilaterally;
(5) ADN unilaterally and CSN unilaterally;
(6) ADN unilaterally and CSN bilaterally;
(7) ADN bilaterally and CSN unilaterally;
(8) ADN bilaterally and CSN bilaterally;
(9) Left ADN unilaterally;
(10) Left ADN unilaterally and CSN unilaterally; or
(11) Left ADN unilaterally and CSN bilaterally.

In aspects of the present disclosure involving electrode attachment unilaterally, and hence unilateral modulation (e.g. stimulation) of the neural activity, of the ADN and/or CSN (i.e. options (1)-(2), (5)-(8), (9)-(11) above), the left or the right nerve may be modulated. In certain embodiments of the present disclosure involving unilateral modulation of the neural activity, the left ADN may be modulated.

In aspects of the present disclosure involving electrode attachment unilaterally, and hence unilateral modulation (e.g. stimulation) of the neural activity, of both the ADN and CSN (i.e. option (5) or (10) above), the nerves are modulated (e.g. stimulated) ipsilaterally.

In aspects of the present disclosure involving electrode attachment, and hence modulation (e.g. stimulation), of more than one nerve (i.e. options (3)-(8) and (10)-(11) above), the signals may be applied simultaneously or sequentially. In certain aspects, the signals are applied simultaneously. The electrode may attach at a single point or at multiple points to any of these nerves. The multiple points may be at the same site of the nerve. In this aspect of the present disclosure, the multiple points may be positioned on the nerve ≤10 mm apart. Alternatively, the multiple points may be at different sites in the same nerve. In this case, the sites may be y mm apart, wherein y≥1 mm, ≥2 mm, ≥3 mm, ≥4 mm, ≥5 mm, ≥6 mm, ≥7 mm, ≥8 mm, ≥9 mm. Alternatively, y may be ≥10 mm, ≥20 mm or ≥30 mm. In one aspect of the present disclosure, the sites may be ≤10 mm apart, in particular where the at least one electrode attaches unilaterally. For example, modulation (e.g. stimulation) may take place at multiple points along the ADN and/or the CSN.

Microprocessor

The implantable device 106, may comprise a processor, for example microprocessor 113. Microprocessor 113 may be responsible for triggering the beginning and/or end of the signals delivered to the nerve by the at least one neural interfacing element. Optionally, microprocessor 113 may also be responsible for generating and/or controlling the signal parameters.

Microprocessor 113 may be configured to operate in an open-loop fashion, wherein a pre-defined signal (e.g. as described above) is delivered to the nerve at a given periodicity (or continuously) and for a given duration (or indefinitely) with or without an external trigger, and without any control or feedback mechanism. Alternatively, microprocessor 113 may be configured to operate in a closed loop fashion, wherein a signal is applied based on a control or feedback mechanism. As described elsewhere herein, the external trigger may be an external controller 101 operable by the operator to initiate delivery of a signal.

A feedback mechanism useful with the present disclosure may involve a processor determining the mean arterial blood pressure, which may optionally compare this value with the normal value. For example, the normal mean arterial blood pressure of a subject may be about 80 mmHg. A subject may have a high mean arterial blood pressure, such as about 120 mmHg.

In some aspects of the present disclosure, the system of the present disclosure can be configured to titrate the amount of total intensity of the signal to be received by the nerve in an open loop (e.g. by an operator) or in a closed loop fashion (e.g. by involving a feedback mechanism for determining the mean arterial blood pressure of the subject). For example, depending on the resulting mean arterial pressure of the subject following a first time period of signal application, the predetermined threshold in a subsequent time period of signal application may be set according to a desired drop in the mean arterial blood pressure in the subject. For example, the predetermined threshold for a first time period of signal application may be set at a total intensity that would produce a 30 mmHg drop in mean arterial blood pressure, and the predetermined threshold for a second time period of signal application may be set at a total intensity that would produce a different drop (e.g. a 10 mmHg drop) in mean arterial blood pressure.

In some aspects of the present disclosure, the system can be configured to deliver an electrical signal when a certain drop in mean arterial blood pressure is detected. The amount of drop in mean arterial pressure that may trigger the application of an electrical signal is described elsewhere herein. The initiation of electrical signal delivery can be triggered in an open loop or closed loop fashion, as explained herein.

Microprocessor 113 of the implantable device 106 may be constructed so as to generate, in use, a preconfigured and/or operator-selectable signal that is independent of any input. In some aspects of the present disclosure, however, microprocessor 113 is responsive to an external signal, for example, information (e.g. data) pertaining to one or more physiological parameters of the subject. Microprocessor 113 may be triggered upon receipt of a signal generated by an operator, such as a physician or the subject in which the device 106 is implanted. To that end, the device 106 may be part of a system 100 which additionally comprises an external system 118 comprising a controller 101. An example of such a system 100 is described below with reference to FIG. 2.

External system 118 of the larger system 100 is external to the internal system 106 and external to the subject, and comprises controller 101. Controller 101 may be used for controlling and/or externally powering system 116. To this end, controller 101 may comprise a powering unit 102 and/or a programming unit 103. The external system 118 may further comprise a power transmission antenna 104 and a data transmission antenna 105, as further described below.

The controller 101 and/or microprocessor 113 may be configured to apply any one or more of the above signals to the nerve intermittently or continuously over a certain period of time, as described herein.

In certain aspects of the present disclosure, the signal is applied only when the subject is in a specific state e.g. only when the subject is awake, only when the subject is asleep, prior to and/or after the ingestion of food, prior to and/or after the subject undertakes exercise, etc.

The various aspects of the present disclosure for timing for modulation of neural activity in the nerve can all be achieved using controller 101 in a device of the present disclosure.

Other Components of the System Including the Implantable Device

In addition to the aforementioned at least one electrode 108 and microprocessor 113, the device 106 may comprise one or more of the following components: implantable transceiver 110; physiological sensor 111; power source 112; memory 114 (otherwise referred to as a non-transitory computer-readable storage device); and physiological data processing module 115. Additionally or alternatively, the physiological sensor 111; memory 114; and physiological data processing module 115 may be part of a sub-system external to the device 106. Optionally, the external sub-system may be capable of communicating with the device 106, for example wirelessly via the implantable transceiver 110.

In some aspects of the present disclosure, one or more of the following components may be contained in the implantable device 106: power source 112; memory 114; and a physiological data processing module 115.

The power source 112 may comprise a current source and/or a voltage source for providing the power for the signal delivered to the ADN and/or CSN by the at least one neural interfacing element (e.g. electrode 108). The power source 112 may also provide power for the other components of the implantable system 116, such as the microprocessor 113, memory 114, and implantable transceiver 110. The power source 112 may comprise a battery, the battery may be rechargeable.

It will be appreciated that the availability of power is limited in implantable devices, and the present disclosure has been devised with this constraint in mind. The implantable system 116 may be powered by inductive powering or a rechargeable power source.

Memory 114 may store power data and data pertaining to the one or more physiological parameters from internal device 116. For instance, memory 114 may store data pertaining to one or more signals indicative of the one or more physiological parameters detected by physiological sensor 111, and/or the one or more corresponding physiological parameters determined via physiological data processing module 115. In addition or alternatively, memory 114 may store power data and data pertaining to the one or more physiological parameters from external system 118 via the implantable transceiver 110. To this end, the implantable transceiver 110 may form part of a communication subsystem of the system 100, as is further discussed below.

Physiological data processing module 115 is configured to process one or more signals indicative of one or more physiological parameters detected by the physiological sensor 111, to determine one or more corresponding physiological parameters. Physiological data processing module 115 may be configured for reducing the size of the data pertaining to the one or more physiological parameters for storing in memory 114 and/or for transmitting to the external system via implantable transceiver 110. Implantable transceiver 110 may comprise an one or more antenna(e). The implantable transceiver 100 may use any suitable signaling process such as RF, wireless, infrared and so on, for transmitting signals outside of the body, for instance to system 100 of which the device 116 is one part.

Alternatively or additionally, physiological data processing module 115 may be configured to process the signals indicative of the one or more physiological parameters and/or process the determined one or more physiological parameters to determine the evolution of the disease in the subject. In such case, the system 116, in particular the implantable device 106, will include a capability of calibrating and tuning the signal parameters based on the one or more physiological parameters of the subject and the determined evolution of the disease in the subject.

The physiological data processing module 115 and the at least one physiological sensor 111 may form a physiological sensor subsystem, also known herein as a detector, either as part of the system 116, part of the implantable device 106, or external to the system.

Physiological sensor 111 comprises one or more sensors, each configured to detect a signal indicative of one of the one or more physiological parameters described above. For example, the physiological sensor 110 is configured for one or more of: detecting the heart rate using a heart rate monitor, detecting electrical activity of the heart and/or heart rhythm using an electrical sensor (e.g. an ECG recorder); detecting blood pressure (e.g. arterial blood pressure) using a pressure sensor; detecting neural activity of a nerve using an electrical sensor; obtaining a neurogram by magnetic resonance neurography using magnetic resonance scanner; or a combination thereof.

The physiological parameters determined by the physiological data processing module 115 may be used to trigger the microprocessor 113 to deliver a signal of the kinds described above to the nerve using the at least one neural interfacing element (e.g. electrode 108). Upon receipt of the signal indicative of a physiological parameter received from physiological sensor 111, the physiological data processor 115 may determine the physiological parameter of the subject, and the evolution of the disease, by calculating in accordance with techniques known in the art. For instance, if a signal indicative of excessive increase in the arterial blood pressure is detected, the processor may trigger delivery of a signal which reduces the arterial blood pressure, as described elsewhere herein.

The memory 114 may store physiological data pertaining to normal levels of the one or more physiological parameters. The data may be specific to the subject into which the system 116 is implanted, and gleaned from various tests known in the art. Upon receipt of the signal indicative of a physiological parameter received from physiological sensor 111, or else periodically or upon demand from physiological sensor 111, the physiological data processor 115 may compare the physiological parameter determined from the signal received from physiological sensor 111 with the data pertaining to a normal level of the physiological parameter stored in the memory 114, and determine whether the received signals are indicative of insufficient or excessive of a particular physiological parameter, and thus indicative of the evolution of the disease in the subject.

The system 116 and/or implantable device 106 may be configured such that if and when an insufficient or excessive level of a physiological parameter is determined by physiological data processor 115, the physiological data processor 115 triggers delivery of a signal to the ADN and/or CSN by the at least one neural interfacing element (e.g. electrode 108), in the manner described elsewhere herein. For instance, if physiological parameter indicative of worsening of any of the physiological parameters and/or of the disease is determined, the physiological data processor 115 may trigger delivery of a signal which dampens secretion of the respective biochemical, as described elsewhere herein. Particular physiological parameters relevant to the present disclosure are described above. When one or more signals indicative of one or more of these physiological parameters are received by the physiological data processor 115, a signal may be applied to the nerve via the at least one neural interfacing element (e.g. electrode 108).

In some aspects of the present disclosure, controller 101 may be configured to make adjustments to the operation of the system 116. For instance, it may transmit, via a communication subsystems (discussed further below), physiological parameter data pertaining to a normal arterial blood pressure. The data may be specific to the patient into which the device is implanted. The controller 101 may also be configured to make adjustments to the operation of the power source 112, signal generator 117 and processing elements 113, 115 and/or neural interfacing elements in order to tune the signal delivered to the ADN and/or CSN nerve by the neural interface.

As an alternative to, or in addition to, the ability of the system 116 and/or implantable device 106 to respond to physiological parameters of the subject, the microprocessor 113 may be triggered upon receipt of a signal generated by an operator (e.g. a physician or the subject in which the system 116 is implanted). To that end, the system 116 may be part of a system 100 which comprises external system 118 and controller 101, as is further described below.

System Including Implantable Device

With reference to FIG. 2, the implantable device 106 of the present disclosure may be part of a system 100 that includes a number of subsystems, for example the system 116 and the external system 118. The external system 118 may be used for powering and programming the system 116 and/or the implantable device 106 through human skin and underlying tissues. The implantable device 106 delivering a signal according to the present disclosure may be configured either externally or internally.

The external subsystem 118 may comprise, in addition to controller 101, one or more of: a powering unit 102, for wirelessly recharging the battery of power source 112 used to power the implantable device 106; and, a programming unit 103 configured to communicate with the implantable transceiver 110. The programming unit 103 and the implantable transceiver 110 may form a communication subsystem. In some aspects of the present disclosure, powering unit 102 is housed together with programing unit 103. In other aspects of the present disclosure, these elements can be housed in separate devices.

The external subsystem 118 may also comprise one or more of: power transmission antenna 104; and data transmission antenna 105. Power transmission antenna 104 may be configured for transmitting an electromagnetic field at a low frequency (e.g., from 30 kHz to 10 MHz). Data transmission antenna 105 may be configured to transmit data for programming or reprogramming the implantable device 10 106, and may be used in addition to the power transmission antenna 104 for transmitting an electromagnetic field at a high frequency (e.g., from 1 MHz to 10 GHz). The temperature in the skin will not increase by more than 2 degrees Celsius above the surrounding tissue during the operation of the power transmission antenna 104. The at least one antennae of the implantable transceiver 110 may be configured to receive power from the external electromagnetic field generated by power transmission antenna 104, which may be used to charge the rechargeable battery of power source 112.

The power transmission antenna 104, data transmission antenna 105, and the at least one antennae of implantable transceiver 110 have certain characteristics such a resonant frequency and a quality factor (Q). One implementation of the antenna(e) is a coil of wire with or without a ferrite core forming an inductor with a defined inductance. This inductor may be coupled with a resonating capacitor and a resistive loss to form the resonant circuit. The frequency is set to match that of the electromagnetic field generated by the power transmission antenna 105. A second antenna of the at least one antennae of implantable transceiver 110 can be used in system 116 for data reception and transmission from/to the external system 118. If more than one antenna is used in the system 116, these antennae are rotated 30 degrees from one another to achieve a better degree of power transfer efficiency during slight misalignment with the with power transmission antenna 104.

External system 118 may comprise one or more external body-worn physiological sensors 121 (not shown) to detect signals indicative of one or more physiological parameters. The signals may be transmitted to the system 116 via the at least one antennae of implantable transceiver 110. Alternatively or additionally, the signals may be transmitted to the external system 116 and then to the system 116 via the at least one antennae of implantable transceiver 110. As with signals indicative of one or more physiological parameters detected by the implanted physiological sensor 111, the signals indicative of one or more physiological parameters detected by the external sensor 121 may be processed by the physiological data processing module 115 to determine the one or more physiological parameters and/or stored in memory 114 to operate the system 116 in a closed loop fashion. The physiological parameters of the subject determined via signals received from the external sensor 121 may be used in addition to alternatively to the physiological parameters determined via signals received from the implanted physiological sensor 111.

For example, in a particular aspect of the present disclosure a detector external to the implantable device may include a non-invasive blood flow monitor, such as an ultrasonic flowmeter and/or a non-invasive blood pressure monitor, and determining changes in physiological parameters, in particular the physiological parameters described above. As explained above, in response to the determination of one or more of these physiological parameters, the detector may trigger delivery of signal to the ADN and/or CSN by the at least one neural interfacing element (e.g. electrode 108), or may modify the parameters of the signal being delivered or a signal to be delivered to the ADN and/or CSN by the at least one neural interfacing element in the future.

The system 100 may include a safety protection feature that discontinues the electrical stimulation of ADN and/or CSN in the following exemplary events: abnormal operation of the system 116 (e.g. overvoltage); abnormal readout from an implanted physiological sensor 111 (e.g. temperature increase of more than 2 degrees Celsius or excessively high or low electrical impedance at the electrode-tissue interface); abnormal readout from an external body-worn physiological sensor 121 (not shown); or abnormal response to stimulation detected by an operator (e.g. a physician or the subject). The safety precaution feature may be implemented via controller 101 and communicated to the system 116, or internally within the system 116.

The external system 118 may comprise an actuator 120 (not shown) which, upon being pressed by an operator (e.g. a physician or the subject), will deliver a signal, via controller 101 and the respective communication subsystem, to trigger the microprocessor 113 of the system 116 to deliver a signal to the nerve by the at least one neural interfacing element (e.g. electrode 108).

System 100 of the present disclosure, including the external system 118, but in particular system 116, may be made from, or coated with, a biostable and biocompatible material. This means that the device is both protected from damage due to exposure to the body's tissues and also minimizes the risk that the device elicits an unfavorable reaction by the host (which could ultimately lead to rejection). The material used to make or coat the device should ideally resist the formation of biofilms. Suitable materials include, but are not limited to, poly(p-xylylene) polymers (known as Parylenes) and polytetrafluoroethylene.

The implantable device 116 of the present disclosure will generally weigh less than 50 g.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the present disclosure.

The term “about” in relation to a numerical value x is optional and means, for example, x±10%.

Unless otherwise indicated each aspect of the present disclosure as described herein may be combined with another aspect of the present disclosure as described herein.

### BRIEF DESCRIPTION OF THE DRAWINGS

FIG. 1 is a schematic diagram of aortic and carotid baroreceptor nerve terminals and nerve trunks [1]. This diagram illustrates the relative anatomical positions of aortic and carotid baroreceptors nerve terminals, their nerve fibers and their somata regions. Aortic baroreceptor nerve terminals are located in the aortic arch. The afferent nerve trunk is the aortic depressor nerve. Soma are in the nodose ganglia (NG). Carotid baroreceptors are positioned in the internal carotid artery next to the carotid bifurcation. Its afferent nerve is the carotid sinus nerve. The soma are located within the petrosal ganglia (PG).

FIG. 2 is a block diagram illustrating elements of a system for performing electrical modulation in ADN and/or CSN according to the present disclosure.

FIGS. 3A and 3B show the circadian rhythms in mean arterial blood pressure (MAP; FIG. 3A) and in heart rate (HR; FIG. 3B) in conscious freely-moving adult (16-week old) Wistar-Kyoto rats (WKY) and Spontaneously Hypertensive rats (SHR). The data are presented as the mean±SEM. There were 18 rats in each group.

FIGS. 4A and 4B show percentage changes where FIG. 4A shows mean arterial blood pressure (MAP) and FIG. 4B shows heart rate (HR) elicited by the electrical stimulation (3V, 1 mA, 2-ms pulse length for 5 sec) of the left aortic depressor nerve in freely-moving 16-week old Wistar Kyoto (WKY) rats and Spontaneously Hypertensive rats (SHR). The data are presented as mean±SEM. There were 12 rats in each group. *P<0.05, significant change from Pre. †P<0.05, 2.5 Hz versus 1 Hz.

FIGS. 5A and 5B show the percentage changes where FIG. 5A shows mean arterial blood pressure (MAP) and FIG. 5B shows heart rate (HR) elicited by electrical stimulation (3V, 1 mA, 2 ms pulse length for 5 sec) of left carotid sinus nerve in conscious 16-week old Wistar Kyoto (WKY) rats and Spontaneously Hypertensive rats (SHR). The data are presented as mean±SEM. There were 12 rats in each group. *P<0.05, significant change from Pre. †P<0.05, 2.5 Hz versus 1 Hz.

FIGS. 6A and 6B show the percentage changes in minute ventilation (MV) elicited by electrical stimulation (3V, 1 mA, 2-ms pulse length for 5 sec) where FIG. 6A is of the left aortic depressor nerve and FIG. 6B is of the left carotid sinus nerve in freely-moving 16-week old Wistar Kyoto (WKY) rats and Spontaneously Hypertensive rats (SHR). The data are presented as mean±SEM. There were 12 rats in each group. *P<0.05, significant change from Pre. †P<0.05, 2.5 Hz versus 1 Hz.

FIGS. 7A and 7B show the circadian rhythms in mean arterial blood pressure (MAP; FIG. 7A) and heart rate (HR; FIG. 7B) in freely-moving 16-week old Spontaneously Hypertensive rats (SHR), which received sham electrical stimulations (ES) of the aortic depressor nerve (SHR—sham) or actual episodes of 1 Hz electrical stimulation (for each period of ES, 12 episodes of stimulation at 3V, 1 mA, 2-ms pulse length for 5 sec, each episode separated by 1 min). The data are presented as mean±SEM. There were 12 rats in each group.

FIG. 8A shows the western blot analyses of Enac protein in nodose ganglia of 16 week old WKY and SHR. Data are mean±SEM. There were 18 rats in each group in A, and 12 rats in each group in B and C. *P<0.05, Stimulation versus Control.

FIGS. 8B and 8C show the western blot analyses of Enac protein in aortic arches of 16 week old WKY and SHR. Data are mean±SEM. There were 12 rats in each group. *P<0.05, Stimulation versus Control.

FIGS. 9A-9C show the baseline mean arterial blood pressures (MAP) in Spontaneously Hypertensive rats (SHR) immediately before they received episodes of electrical stimulation of the right ADN on days 7, 14 and 21 post-surgery. There were 4 male SHR in the group. The data are presented as mean±SEM. *P<0.05, Day 14 or Day 21 versus Day 7. †P<0.05, Day 21 versus Day 14 (FIGS. 9B and 9C).

FIGS. 10A-10C show the falls in mean arterial blood pressure (MAP) in Spontaneously Hypertensive rats (SHR) elicited by electrical stimulation of the right ADN on days 7, 14 and 21 post-surgery. There were 4 male SHR in the group. The data are presented as mean±SEM. *P<0.05, Day 14 or Day 21 versus Day 7. †P<0.05, Day 21 versus Day 14.

FIGS. 11A-11C show the time-course of decreases in mean arterial blood pressure (MAP) in Spontaneously Hypertensive rats (SHR) elicited by electrical stimulation of the right ADN on days 7, 14 and 21 post-surgery. There were 4 male SHR in the group. The data are presented as mean±SEM. *P<0.05, Day 14 or Day 21 versus Day 7. †P<0.05, Day 21 versus Day 14.

FIGS. 12A and 12B shows the body weights of the rats during the experiment. The data are presented as mean±SEM. *P<0.05, Day 14 or Day 21 versus Day 7. †P<0.05, Day 21 versus Day 14.

FIGS. 13A-13C show the effects of electrical stimulation of one aortic depressor nerve (ADN-S) on frequency of breathing and disordered breathing Index (DBI) values of freely-moving sham-operated Sprague-Dawley rats. There were 9 rats in each group. The data is presented as mean±SEM. *P<0.05, significant response. †P<0.05, ADN-S versus Sham-stimulation. Stimulation immediately post H-H challenge.

FIGS. 14A-14C show the effects of electrical stimulation of one aortic depressor nerve (ADN-S) on frequency of breathing and disordered breathing Index (DBI) values of freely-moving sham-operated Sprague-Dawley rats. There were 9 rats in each group. The data is presented as mean±SEM. *P<0.05, significant response. †P<0.05, ADN-S versus Sham-stimulation. Simulation at 5 min post H-H challenge.

FIG. 15 shows the mean arterial blood pressure (MAP) values of sham-operated Sprague-Dawley rats and those with bilateral aortic depressor nerve transection (ADNX) during the light and dark cycles. There were 10 rats in each group. The data is presented as mean±SEM.

FIG. 16 shows the mean arterial blood pressure (MAP) values of sham-operated Sprague-Dawley rats and those with bilateral aortic depressor nerve transection (ADNX) during the light and dark cycles. There were 10 rats in each group. The data is presented as mean±SEM.

FIG. 17 shows the disordered breathing index (DBI) values of sham-operated Sprague-Dawley rats and those with bilateral aortic depressor nerve transection (ADNX) during the light and dark cycles. There were 10 rats in each group. The data is presented as mean±SEM.

FIGS. 18A and 18B show a sample data trace showing blood pressure (BP), heart rate (HR), femoral blood flow (FBF) and mesenteric blood flow (MBF) responses to right aortic depressor nerve stimulation in urethane-anesthetized male Sprague Dawley (SD) rats. The stimulations were performed using bipolar silver stimulating electrodes (1-20 Hz, 0.4 mA, 0.2 ms for 20 s).

FIG. 19 shows a sample data trace showing blood pressure (BP), heart rate (HR) and femoral blood flow (FBF) responses to left aortic depressor nerve stimulation in urethane-anesthetized male and female Sprague Dawley (SD) rats. Stimulations were performed using bipolar silver stimulating electrodes (1-20 Hz, 0.4 mA, 0.2 ms for 20 s).

FIG. 20 shows the mean arterial blood pressure (MAP) responses elicited by electrical stimulation (1-20 Hz, 0.4 mA, 0.2 ms for 20 s) of the left or the right aortic depressor nerve (ADN) in urethane-anesthetized male and female Sprague-Dawley rats. Stimulation was performed using bipolar silver stimulating electrodes. Data presented as mean±SEM (n=6 rats in each group).

FIG. 21 shows the effect of modifying pulse width, amplitude and frequency of left aortic depressor nerve (ADN) stimulation on changes in peak mean arterial pressure (MAP) in anesthetized male spontaneously hypertensive rats (SHR) (n=4).

FIGS. 22A-22B2 show the mean arterial pressure (MAP) responses where FIG. 22A, FIGS. 22A1 and 22A2 are to low (5 Hz) frequency (FIGS. 22B, 22B1 and 22B2 are to high (15 Hz) frequency), continuous (20 s) versus intermittent (5 s on/3 s off and 5 s on/3 s off for 20 s) left aortic depressor nerve (ADN) stimulation (0.4 mA, 0.2 ms) in 25-26 weeks old male SHR (n=8), FIGS. 22A & 22B show time course analysis calculated as 5 s bins and plotted as 40 s baseline and 80 s after stimulation; FIGS. 22A1 & 22B1 show peak changes in MAP relative to baseline; and FIGS. 22A2 & 2B2 show differences in peak changes evoked by intermittent versus continuous stimulation. *P≤0.05.

FIGS. 23A-23B2 show the heart rate (HR) responses where FIGS. 23A, 23A1 and 23A2 are to low (5 Hz) frequency (FIGS. 23B, 23B1 and 23B2 are to high (15 Hz) frequency), continuous (20 s) versus intermittent (5 s on/3 s off and 5 s on/3 s off for 20 s) left aortic depressor nerve (ADN) stimulation (0.4 mA, 0.2 ms) in 25-26 weeks old male SHR (n=8), FIGS. 23A & 23B show time course analysis calculated as 5 s bins and plotted as 40 s baseline and 80 s after stimulation; FIGS. 23A1 & 23B1 show peak changes in HR relative to baseline; and FIGS. 23A2 & 23B2 show differences in peak changes evoked by intermittent versus continuous stimulation. *P≤0.05.

FIGS. 24A-24B2 show he femoral vascular resistance (FVR) responses where FIGS. 24A, 24A1 and 24A2 are to low (5 Hz) frequency (FIGS. 24B, 24B1 and 24B2 are to high (15 Hz) frequency), continuous (20 s) versus intermittent (5 s on/3 s off and 5 s on/3 s off for 20 s) left aortic depressor nerve (ADN) stimulation (0.4 mA, 0.2 ms) in 25-26 weeks old male SHR (n=8); FIGS. 24A & 24B show time course analysis calculated as 5 s bins and plotted as 40 s baseline and 80 s after stimulation; FIGS. 24A1 & 24B1 show peak changes in FVR relative to baseline; and FIGS. 24A2 & 24B2 show differences in peak changes evoked by intermittent versus continuous stimulation. *P≤0.05.

FIGS. 25A-25B2 show the mesenteric vascular resistance (MVR) responses where FIGS. 25A, 25A1 and 25A2 are to low (5 Hz) frequency (FIGS. 25B, 25B1 and 25B2 are to high (15 Hz) frequency), continuous (20 s) versus intermittent (5 s on/3 s off and 5 s on/3 s off for 20 s) left aortic depressor nerve (ADN) stimulation (0.4 mA, 0.2 ms) in 25-26 weeks old male SHR (n=8). FIGS. 25A & 25B show time course analysis calculated as 5 s bins and plotted as 40 s baseline and 80 s after stimulation; FIGS. 25A1 & 25B1 show peak changes in MVR relative to baseline; and FIGS. 25A2 & 25B2 show differences in peak changes evoked by intermittent versus continuous stimulation. *P≤0.05.

FIG. 26 shows the percent changes in mean arterial blood pressure (MAP) elicited by a 30 second burst of electrical stimulation (0.2, 0.5 or 1.0 ms, 5 Hz, 1 mA) of the left (L), right (R) or both (LR) cervical sympathetic chains (CSC, left panels) or L, R or LR superior cervical ganglia (SCG, right panels). The CSC and SCG studies were done in different rats (n=6 per group). Data are shown as mean±SEM.

FIG. 27 shows the change in mean arterial pressure (MAP), heart rate (HR), mesenteric blood flow (MBF) and femoral blood flow (FBF) upon unilateral left ADN stimulation (1, 2.5, 5, 10, 20 and 40 Hz, 0.4 mA, 0.2 ms for 20 s) in sodium pentobarbital-anaesthetized male spontaneously hypertensive rats.

FIGS. 28A-28D show the frequency dependent reductions where FIG. 28A is mean arterial pressure (MAP), FIG. 28B is heart rate (HR), FIG. 28C is mesenteric blood flow (MBF) and FIG. 28D is femoral blood flow (FBF) upon left or right unilateral or bilateral ADN stimulation in male spontaneously hypertensive rats. Mean data ±S.E.M of 6-9 animals. ^{a}P≤0.05, left vs. right ADN, ^{b}P≤0.05, left vs. bilateral ADN and ^{c}P≤0.05, right vs. bilateral ADN analyzed by 2-way ANOVA followed by Tukey's posthoc.

FIG. 29 shows mean arterial pressure (MAP), heart rate (HR), mesenteric blood flow (MBF) and femoral blood flow (FBF) upon unilateral left ADN stimulation (1, 2.5, 5, 10, 20 and 40 Hz, 0.4 mA, 0.2 ms for 20 s) in urethane-anaesthetized male Sprague Dawley rats.

FIGS. 30A-30D show frequency dependent reductions where FIG. 30A is mean arterial pressure (MAP), FIG. 30B is heart rate (HR), FIG. 30C mesenteric blood flow (MBF) and FIG. 30D is femoral blood flow (FBF) upon left or right unilateral or bilateral ADN stimulation in male Sprague Dawley rats. Mean data ±S.E.M of 3-5 animals. ^{b}P≤0.05, left vs. bilateral ADN analyzed by 2-way ANOVA followed by Tukey's post hoc.

FIGS. 31A-31D show representative stained (methylene blue, toluidine blue and hematoxylin) vaginal smears collected from female spontaneously hypertensive rats (SHR) illustrating all 4 stages of oestrus cycle female spontaneously hypertensive rats.

FIG. 32 shows mean arterial pressure (MAP), heart rate (HR), mesenteric blood flow (MBF) and femoral blood flow (FBF) upon unilateral left ADN stimulation (1, 2.5, 5, 10, 20 and 40 Hz, 0.4 mA, 0.2 ms for 20 s) in sodium pentobarbital-anaesthetized female spontaneously hypertensive rats.

FIGS. 33A-33D show frequency dependent reductions where FIG. 33A is mean arterial pressure (MAP), FIG. 33B is heart rate (HR), FIG. 33C is mesenteric blood flow (MBF) and FIG. 33D is femoral blood flow (FBF) upon left or right unilateral or bilateral ADN stimulation in female spontaneously hypertensive rats. Mean data ±S.E.M of 5-8 animals. ^{c}P≤0.05, right vs. bilateral ADN analyzed by 2-way ANOVA followed by Tukey's post hoc.

FIGS. 34A-34D show representative stained (methylene blue, toluidine blue and hematoxylin) vaginal smears collected from female spontaneously hypertensive rats (SHR) illustrating all 4 stages of oestrus cycle in Sprague Dawley rats.

FIG. 35 shows mean arterial pressure (MAP), heart rate (HR), mesenteric blood flow (MBF) and femoral blood flow (FBF) upon unilateral left ADN stimulation (1, 2.5, 5, 10, 20 and 40 Hz, 0.4 mA, 0.2 ms for 20 s) in urethane-anaesthetized female Sprague Dawley (SD) rats.

FIGS. 36A-36D show frequency dependent reductions where FIG. 36A is mean arterial pressure (MAP), FIG. 36B is heart rate (HR), FIG. 36C is mesenteric blood flow (MBF) and FIG. 36D femoral blood flow (FBF) upon left or right unilateral or bilateral ADN stimulation in female Sprague Dawley (SD) rats. Mean data ±S.E.M of 6-7 animals. ^{a}P≤0.05, left vs. right; ^{b}P≤0.05, left vs. bilateral and ADN ^{c}P≤0.05, right vs. bilateral ADN analysed by 2-way ANOVA followed by Tukey's posthoc.

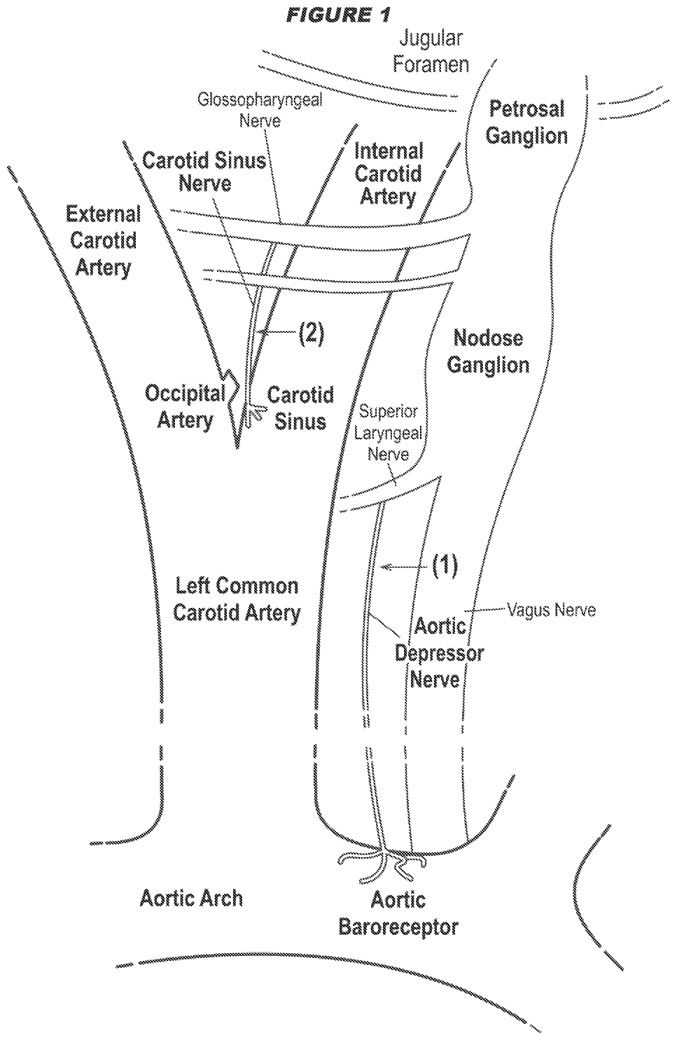

### MODES FOR CARRYING OUT THE PRESENT DISCLOSURE

Study 1

This study investigated whether electrical stimulation of aortic depressor nerves (ADN) in freely-moving Spontaneously Hypertensive rats (SHR) can be a potential therapeutic modality from multiple perspectives including physiology and biochemistry.

Introduction

Baroreceptor afferents emanating from the aortic arch travel within the aortic depressor nerve (ADN) whereas baroafferents emanating from the carotid sinus travel in the carotid sinus nerve (CSN), which also carries chemoafferents from the carotid body [76,77]. In the rat, the ADN has a pure population of baroreceptor afferents3-7 and the electrical stimulation of this nerve is being used to evaluate neural/hemodynamic processes in normotensive and hypertensive rats [78,79,80,81,82].

Baroreceptor afferent sensitivity and baroreceptor reflex-mediated changes in heart rate and sympathetic nerve activity are impaired in adult spontaneously hypertensive rats (SHR) [83,84,85,86,87]. The deficit in baroreflex function lies in the mechanosensitive regions of the peripheral terminals imbedded in vascular smooth muscle [83-85,87]. Electrical stimulation (ES) of baroafferent fibers in the ADN of SHR bypasses the site of impaired baroreceptor mechano-sensory transduction and provides data about the central processing of the afferent input and the properties of central and efferent components of the baroreflex [81,82]. ES allows for precise control of afferent signals transmitted to the nucleus of the tractus solitaries [81,82].

This study investigated ES of ADN and CSN at low frequencies in SHR.

Results

Circadian Rhythms in MAP and Heart Rate

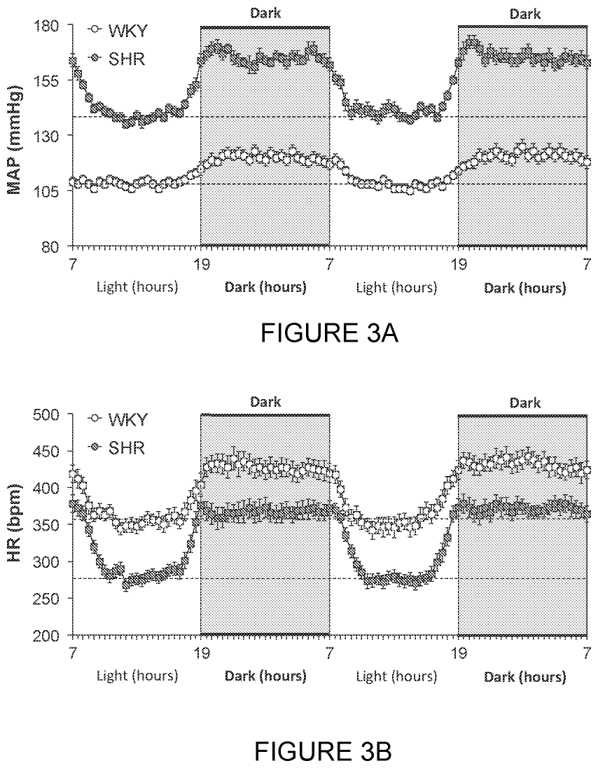

Actual levels of MAP and heart rate of conscious normotensive 16-week old Wistar-Kyoto rats (WKY) and Spontaneously Hypertensive rats (SHR) during the consecutive day-night cycles are shown in FIGS. 3A and 3B. As reported by others [88,89,90,91], MAP and heart rate of WKY and SHR displayed a diurnal rhythm with MAP values being consistently higher during the dark phases and MAP values of SHR being consistently higher than those of the WKY.

Cardiovascular Responses Elicited by ADN Stimulation

Salgado and his colleagues [81,82] employed a relatively high stimulus intensity (1 mA, 2 ms pulses) to activate all fibers in the ADN of conscious normotensive control rats (NCR) and SHR and varied the frequency of stimulation (5-90 Hz) over a wide range to define the full frequency-response relationship. These stimulations were performed during the day-light hours [81,82]. They found that (a) 5 Hz stimulation lowered MAP in NCR and SHR by 25 mmHg whereas in lowered heart rate by 70 beats/min in NCR and 50 beats/min in SHR, and (b) progressively higher frequency ES elicited substantially greater falls in MAP in SHR than in NCR and now equivalent falls in heart rate in both strains.

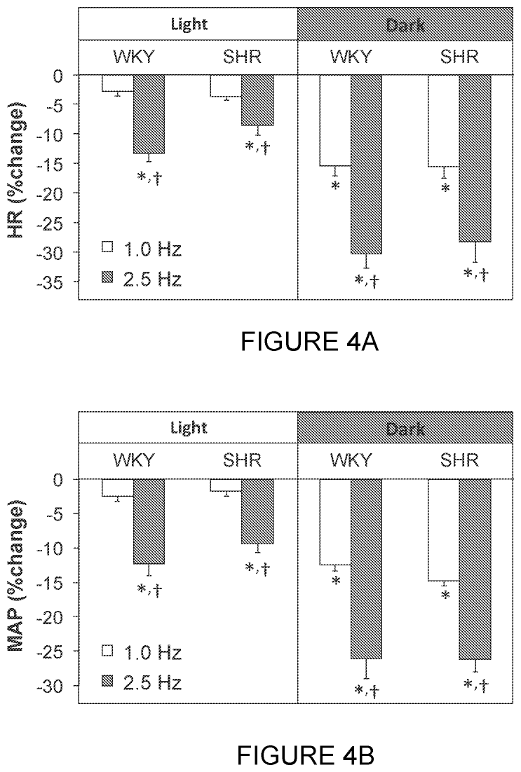

The inventors explored whether the timing of the stimulus over the day-night cycle influences the cardiovascular responses elicited by ES of the ADN in freely-moving WKY and SHR. The inventors used lower frequencies of stimulation (1 and 2.5 Hz) to seek a threshold for the reflex responses. As summarized in FIGS. 4A and 4B, the 1 Hz frequency ES of the left ADN elicited minor responses during the light-cycle (noon-2 PM) in WKY and SHR whereas it elicited more robust responses (similar in WKY and SHR) during the dark-cycle (midnight-2 AM). The 2.5 Hz ES elicited small but observable responses during the light-cycle of similar magnitude in WKY and SHR and substantially greater and equivalent between-group responses during the dark-cycle.

Cardiovascular Responses Elicited by CSN Stimulations

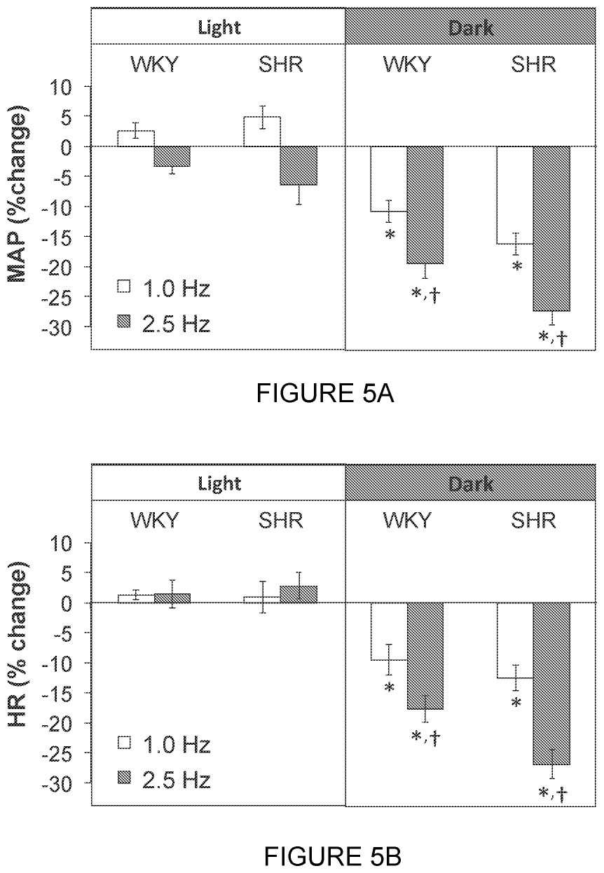

As shown in FIGS. 5A and 5B, neither the 1 nor 2.5 Hz stimulation of the left CSN elicited significant responses when given during the light phase. However, these stimulations elicited robust decreases in MAP and heart rate (2.5 Hz was more effective that 1 Hz stimulation) in both WKY and SHR when applied during the dark phase. The frequency dependent changes in MAP and heart rate were similar in WKY and SHR.

Changes in Minute Ventilation Elicited by ES of the ADN or CSN

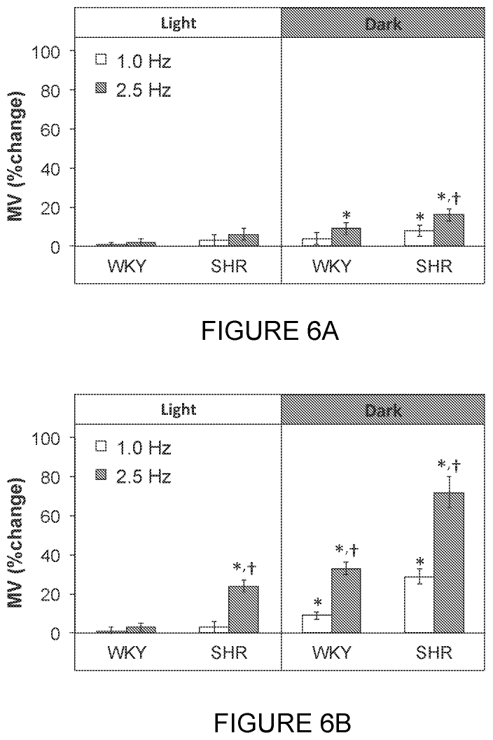

As summarized in FIGS. 6A and 6B, ES of the left ADN elicited minor increases in Minute Ventilation (MV) in conscious WKY or SHR rats. The observable increase in MV elicited by ES of the ADN in WKY and SHR during the dark-cycle is likely baroafferent-driven in response to the falls in MAP [76-78].

In contrast, activation of chemoafferents in the CSN will directly increase MV [76-78]. During the light-cycle, ES of the left CSN at 1 or 2.5 Hz elicited minor increases in MV in WKY rats whereas ES elicited a robust response in SHR. During the dark-cycle, ES of the CSN elicited frequency-dependent increases in MV in WKY and SHR and again the responses were greater in SHR.

ES of the ADN as a Therapeutic Modality

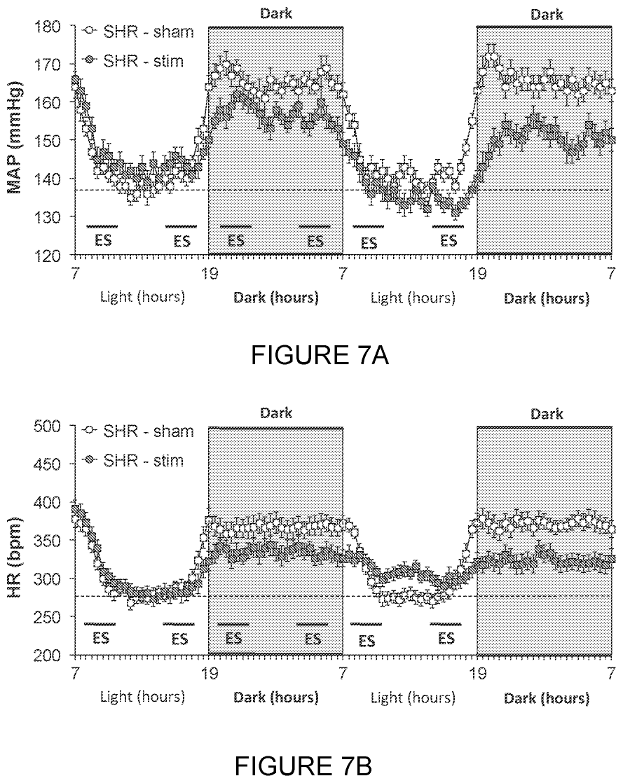

The circadian rhythm in MAP and heart rate in freely-moving 16-week old SHR, which received sham ES of the ADN or actual episodes of 1 Hz ES (12 episodes of stimulation at 3V, 1 mA, 2-ms pulse length for 5 sec, each episode separated by 1 min, for each period of ES) is shown in FIGS. 7A and 7B. The episodes of ES influenced the circadian pattern of both MAP and heart rate especially following the 6th series of ES (second dark-cycle), in which MAP and heart rate were lower than in the non-stimulated SHR.

Vagal Nerve Stimulation Improves Enac Channel Density in the Plasma Membranes of Nodose Ganglion Cell Bodies of SHR:

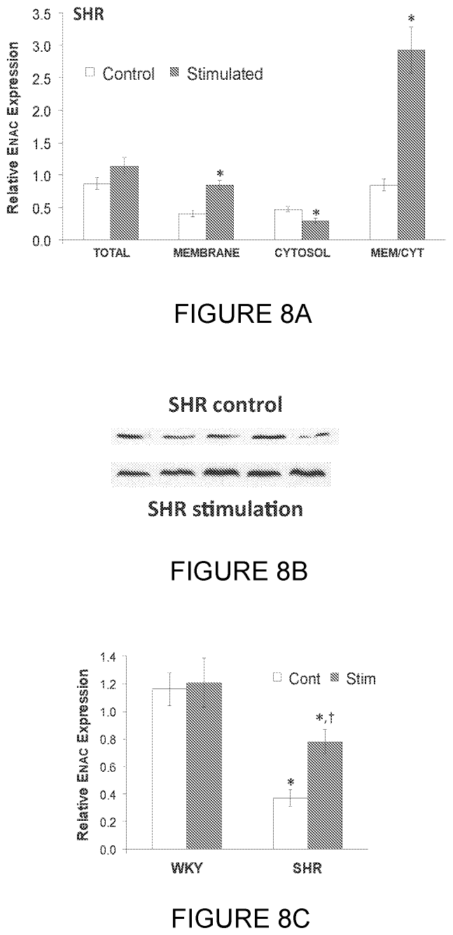

There is substantial evidence that plasma membrane ion-channels of the DEG/epithelial Na+ channel (ENaC) family play a vital role in mechanosensation in and vagal afferents and aortic arch baroafferents [92,93,94]. The inventors applied episodes of 1 Hz ES for 6 consecutive days (12 episodes of stimulation for each session at 3V, 1 mA, 2-ms pulse length for 5 sec, each episode separated by 1 min) to freely-moving SHR. Stimuli were applied during the 60 min period immediately preceding lights off. At the end of the 6th session of ES, the ipsilateral nodose ganglia were removed for Western blot analyses of ENAC protein. As seen in FIG. 8A, the ES protocol improved the disposition of Enac channels in the plasma membranes of nodose ganglion cell bodies, suggesting an ES-induced increase in synthesis and/or diminished rate of degradation by mechanisms yet to be determined.

ADN Stimulation Improves Enac Channel Density in Baroafferent Terminals in Aortic Arch of SHR

Most importantly, aortic arches taken from non-stimulated (control) and ADN stimulated SHR revealed that the ES protocol elicited a substantial improvement of Enac expression within baroafferent nerve terminals by again, mechanisms that are yet to be determined. The results are shown in FIGS. 8B and C.

Study 2

This study investigated the cardiovascular consequences of unilateral stimulation of the right aortic depressor nerve (ADN) in freely-moving Spontaneously Hypertensive rats (SHR). The aim was to determine whether it was possible to intermittently electrically stimulate the right aortic depressor nerve (ADN) of adult male spontaneously hypertensive rats (SHR) for 21 days.

Protocols

The right ADN of 4 adult male SHR was implanted with a Cortec micro-cuff electrode (100 μm). The rats also received a non-occlusive abdominal aorta catheter in order to monitor pulsatile (PP) and mean (MAP) arterial blood pressure. Starting at 7 days post-surgery and continuing each day to 21 days, the rats received three episodes of electrical stimulation (ES, 5 Hz, 8V, 0.5 ms) of 3 min in duration, each separated usually by 15 min beginning at 5 μm. Arterial blood pressure responses to the ADN stimulations were measured on days 7, 14 and 21.

Results

Baseline Arterial Blood Pressures Prior to Each Session of ADN Stimulation

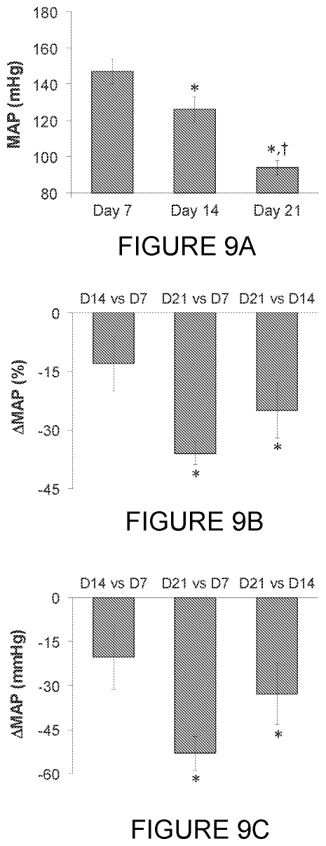

As seen in FIGS. 9A-9C, the brief bursts of ES of the ADN that began on day 7, had a long lasting depressor on MAP that was evident by day 14 and day 21, resting MAP (94±4 mmHg) for these SHR were lower than those of normotensive Wistar-Kyoto rats (n=8) that did not receive ADN stimulations (104±2 mmHg, P<0.05).

Electrical Stimulation Responses

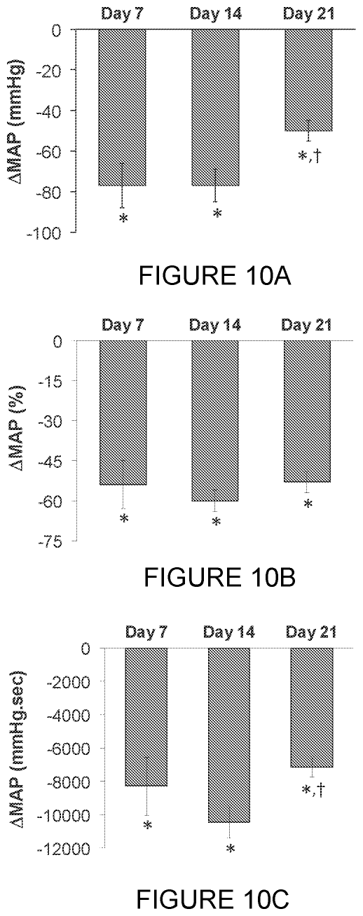

The depressor responses elicited by ES of the ADN on days 7, 14 and 21 are shown in FIGS. 10A-10C. The average of the 3 ES was taken for each rat and the mean±SEM of the group data are presented. As can be seen, ES of the ADN elicited robust decreases in MAP on each day although the magnitude and totality of the responses (area under the curve, bottom right panel) were smaller on day 21 than on days 7 and 14.

Electrical Stimulation—Time-Course

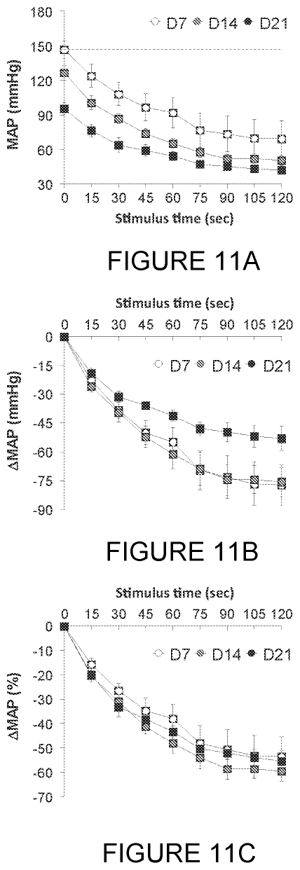

The changes in MAP during elicited by ES of the ADN on days 7, 14 and 21 are shown in FIGS. 11A-11C. The time to reach half-maximal response on Days 7, 14 and 21 were 28.5±2.8, 26.5±1.8 and 21.0±4.2, respectively (P<0.05 for all comparisons).

Body Weights

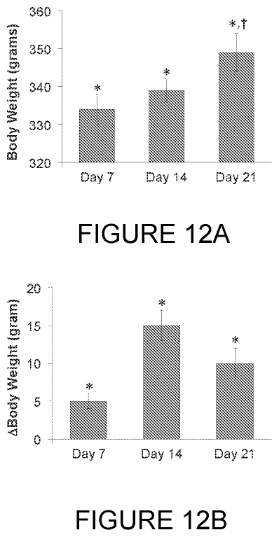

The body weights of the 4 SHR recorded on days 7, 14 and 21 are shown in FIGS. 12A and 12B (values recorded one hour before the ADN stimulations were applied). As can be seen, the rats gained weight at the rate of about 8 grams per week, a value equivalent to non-stimulated SHR.

Summary

These results in SHR show that electrical stimulation of the ADN can be maintained for 21 days, although these 4 represent only 40% of the SHR (n=10) that were attempted.

Study 3

This study investigated the effects of electrical stimulation of left or right ADN on the frequency of breathing, and disordered breathing index in freely-moving Sprague-Dawley rats (SPR).

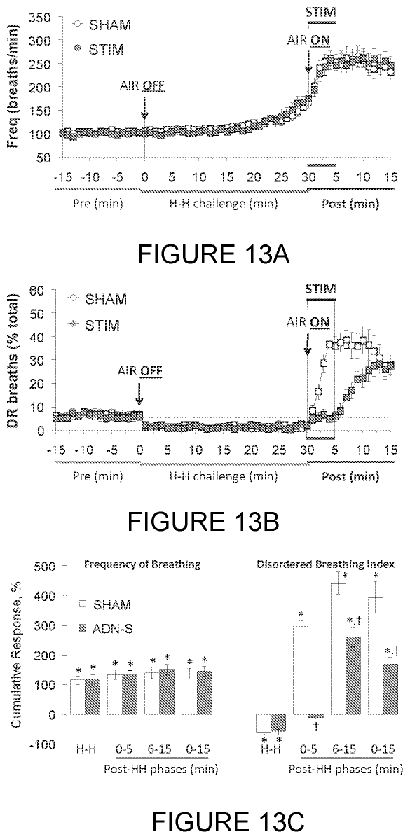

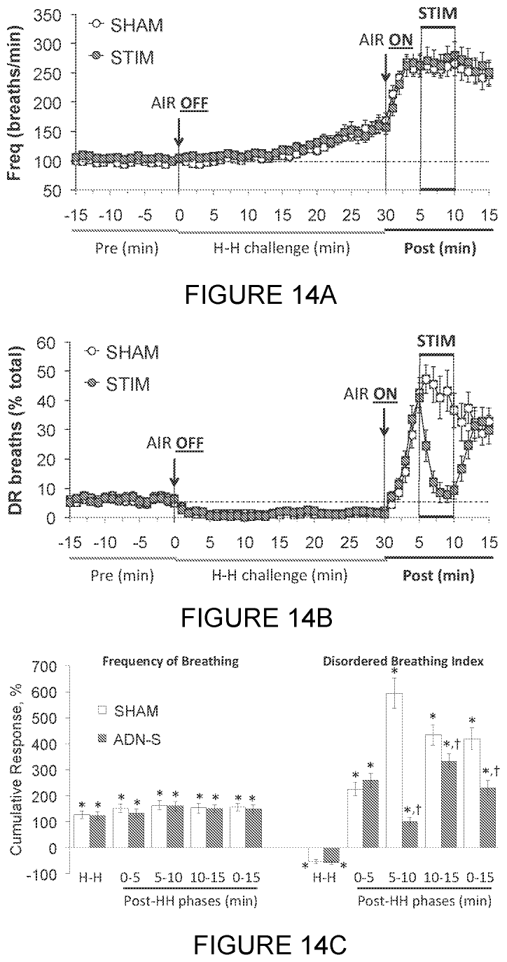

Hypoxic-hypercapnic gas (H-H) challenge (10% 02, 5% CO2) was performed in the rats. The nerve was stimulated immediately post challenge (FIGS. 13A-13C) and at 5 min post challenge (FIGS. 14A-14C).

As shown in FIGS. 13A-13C and 14A-14C, unilateral low intensity electrical stimulation (1 Hz, 8 V, 0.5 msec every alternate 15 sec for 5 min) of left or right and did not affect frequency of breathing but dramatically lowered the disordered breathing index (DBI) in freely-moving Sprague-Dawley rats.

Study 4

This study investigated the effects of bilateral aortic depressor nerve transection (ADNX) on circadian rhythms of mean arterial blood pressure, frequency of breathing, and disordered breathing index in freely-moving sham-operated Sprague-Dawley rats and in ADNX Rats.

Mean Arterial Blood Pressure (MAP)

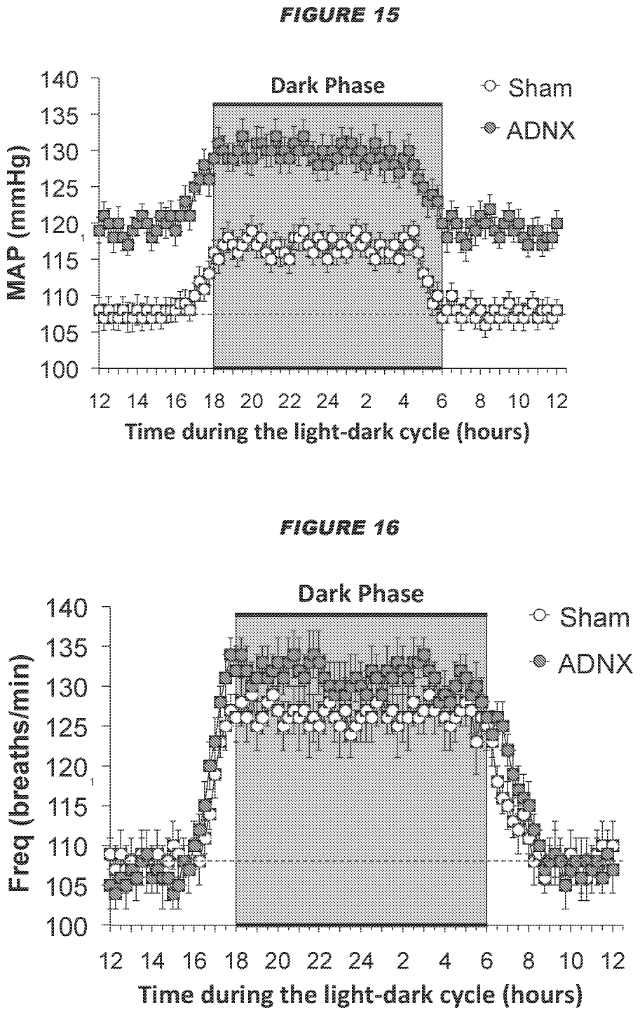

As shown in FIG. 15 and Table 1, freely-moving male adult Sprague-Dawley rats with bilateral ADNX display substantially higher levels of blood pressure during the light and dark cycles than sham-operated controls.

TABLE 1
Average mean arterial pressure
values during the light and dark cycle
Phase of the Light-Dark Cycle

| Group | Light-Cycle | Dark-Cycle |
|---|---|---|
| Sham | 108.2 ± 1.7 mmHg | 116.2 ± 1.8 mmHgª |
| ADNX | 120.2 ± 1.9 mmHg^{b} | 128.9 ± 2.2 mmHg^{a,b} |

ADNX, aortic depressor nerve transection.
The data is presented as mean ± SEM. There were 10 rats in each group.
ªP < 0.05, dark-cycle versus light cycle.
^{b}P < 0.05, ADNX versus Sham.

Frequency of Breathing

As shown in FIG. 16 and Table 2, freely-moving Sprague-Dawley rats with bilateral transection of aortic depressor nerves display similar frequency of breathing values to sham-operated rats during the light and dark cycles.

TABLE 2
Average frequency of breathing
values during the light and dark cycle
Phase of the Light-Dark Cycle

| Group | Light-Cycle | Dark-Cycle |
|---|---|---|
| Sham | 111.4 ± 2.7 breaths/min | 126.5 ± 3.0 breaths/minª |
| ADNX | 112.4 ± 2.6 breaths/min | 131.1 ± 2.8 breaths/minª |

ADNX, aortic depressor nerve transection.
The data is presented as mean □ SEM. There were 10 rats in each group.
ªP < 0.05, dark-cycle versus light cycle.

Disordered Breathing Index

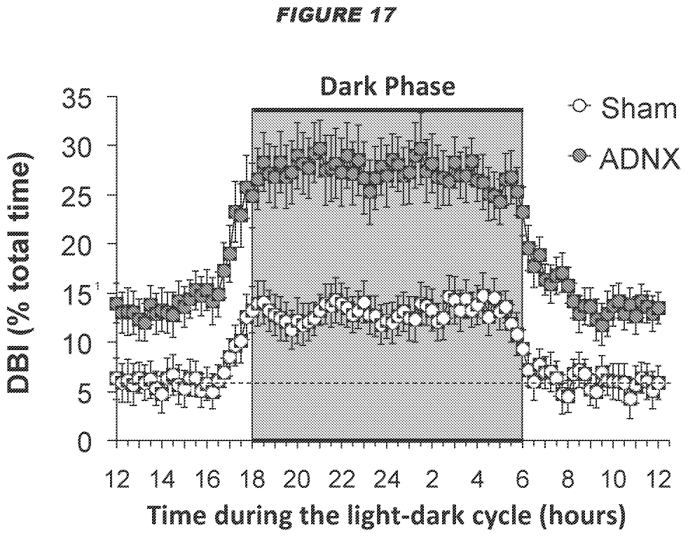

As shown in FIG. 17 and Table 3, freely-moving Sprague-Dawley rats with bilateral transection of aortic depressor nerves display higher disordered breathing indices (DBI) during light and dark cycles than sham-operated rats.

TABLE 3
Average Disordered Breathing values
during the light and dark cycle
Phase of the Light-Dark Cycle

| Group | Light-Cycle | Dark-Cycle |
|---|---|---|
| Sham | 6.5 ± 1.8 mmHg | 13.0 ± 2.0 mmHg^{a} |
| ADNX | 15.2 ± 2.1 mmHg^{b} | 27.3 ± 3.0 mmHg^{a,b} |

ADNX, aortic depressor nerve transection.
The data is presented as mean ± SEM. There were 10 rats in each group.
ªP < 0.05, dark-cycle versus light cycle.
^{b}P < 0.05, ADNX versus Sham.

Study 5

This study investigated the sex differences in cardiovascular responses elicited by electrical stimulation of tandADN in urethane-anesthetized male and female Sprague-Dawley rats.

Results

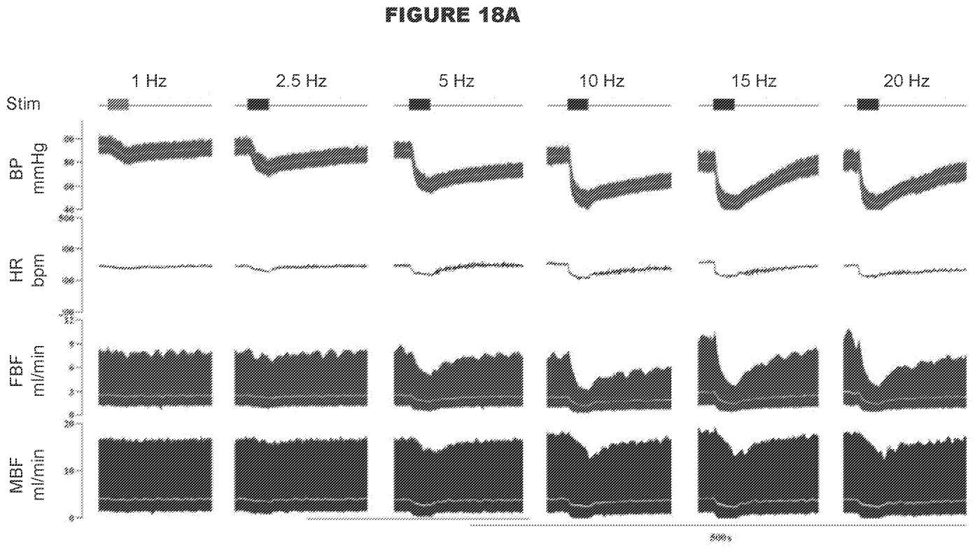

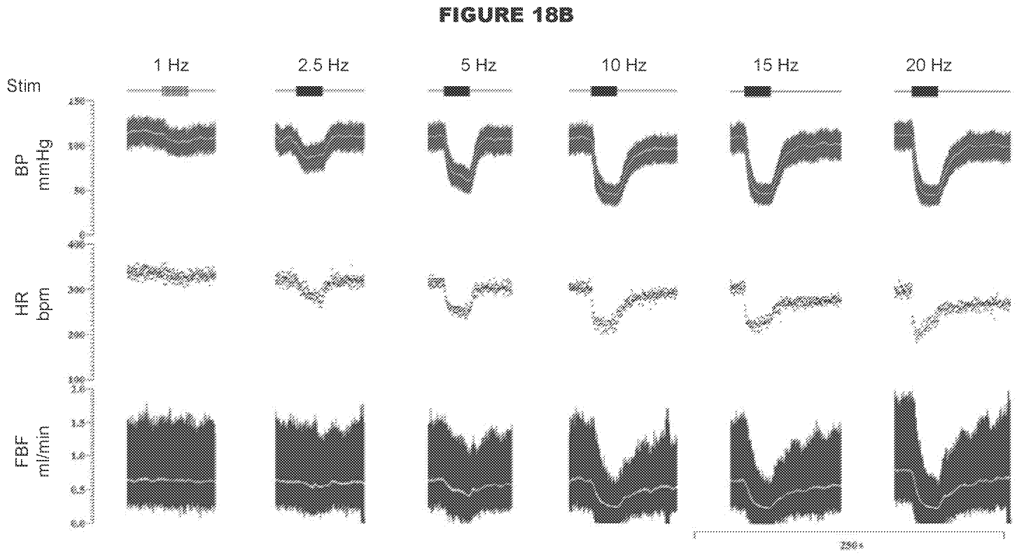

Typical examples of cardiovascular responses elicited by direct electrical stimulation (1-20 Hz, 0.4 mA, 0.2 ms for 20 s) of an aortic depressor nande (ADN) in a male and in a female urethane-anesthetized Sprague-Dawley rat are shown in FIG. 18A and FIG. 18B, respectively.

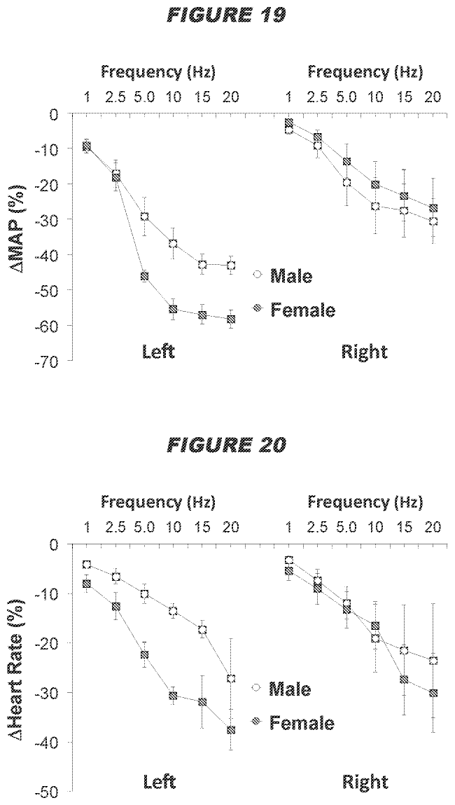

Summaries of the percentage changes in mean arterial blood pressure (MAP) and heart rate (HR) elicited by direct electrical stimulation (1-20 Hz, 0.4 mA, 0.2 ms for 20 and of an ADN in male and female urethane-anesthetized Sprague-Dawley rat are shown in FIG. 19 and FIG. 20, respectively. As can be seen, stimulation and the left ADN in females elicited substantially greater responses than that in male rats. The depressor responses elicited by stimulatiandof the left ADN in males and females were greater than those elicited by theandspective right ADN.

Study 6

This study aimed to identify optimal and andimally disturbing ADN stimulation parameters that would provide a sustained drop in mean arterial pressure (MAP) of ˜30 mmHg in spontaneously hypertensive rats (SHR). This study also aimed to identify potential hemoandamic contributors to ADN stimulation-evoked hypotension in the SHRs.

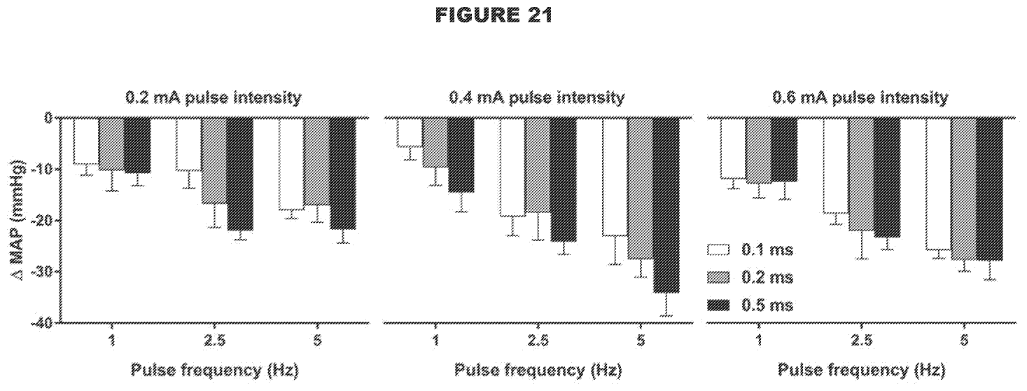

Adult male SHRs (n=4) were anesthetized with urethane (1.2 g/kg i.p.). The SHRs were spontaneously breathing. The mean arterial blood preandre (MAP) in response to ADN stimulation was recorded. The SHRs were stimulated at low ranges of frequencies (1, 2.5 and 5 Hz), pulse amplitudes (0.2, 0.4 and 0.6 mA) and pulse widths (0.1, 0.2 and 0.5 ms) ands shown in FIG. 21, left ADN stimulation in the SHR lowered MAP in a frequency-dependent manner at all pulse amplitudes and widths used. There was no added hypotensive benefit of pulse amplitudes beyond 0.4 mA (maximum MAP drop=˜34 mmHg at 0.4 mA).

It was also found that hypotension was relatively prolonged with higher charge injection resulting in a hypotensive duration of 42 seconds at 0.4 or 0.6 mA versus 32 seconds at 0.2 mA.

Study 7

Adult male 25-26 weeks old SHRs (n=8) were anesthetized with pentobarbital (50 mg/kg i.p. followed by 10 mg/kg i.v. infusion set at 2 ml/h). The SHRs were spontaneously breathing.

The MAP and HR responses to continuous (20 s) and intermittent (5 s on/3 s off and 5 s on/5 s off for 20 s) andolar stimulations of the left ADN at low (5 Hz) and high (15 Hz) pulse frequencies (based on Study 6, a 0.4 mA pulse amplitude and 0.2 ms pulse width were chosen for this study) were recorded. The left femoral artery and superior mesenteric artery blood flows were simultaneously recorded using a transonic blood flow cuff and calculated respective changes in vascular resistance.

Mean Arterial Pressure (MAP) and Heart Rate (HR) Responses

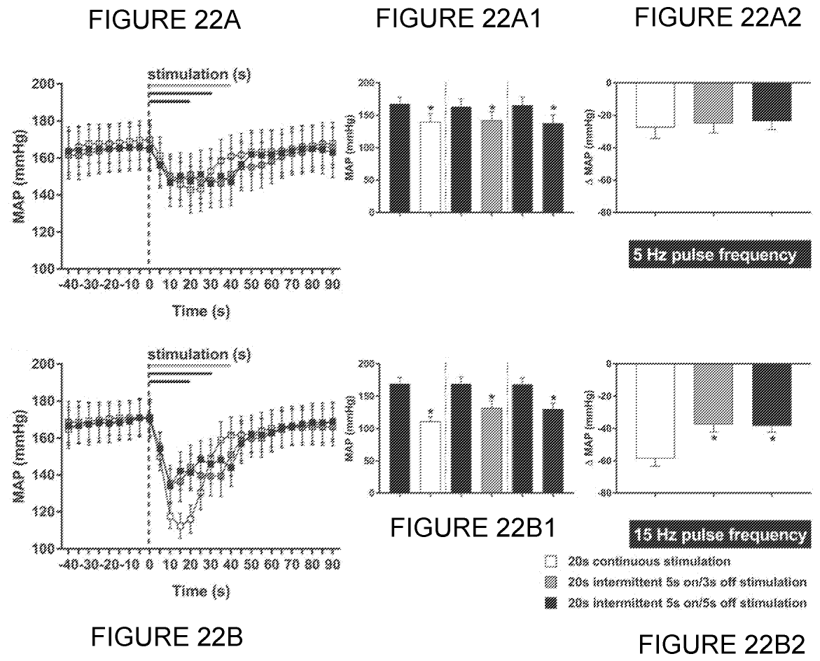

As shown in FIG. 22A (and FIGS. 22A1 and 22A2), intermittent and continuous stimulation of the ADNs produced comparable drop in MAP at the low frequency stimulation.

As shown in FIG. 22B (and FIGS. 22B1 and 22B2), at 15 Hz, intermittent stimulation offered less intense and more acceptable drop in MAP compared to continuous stimulation.

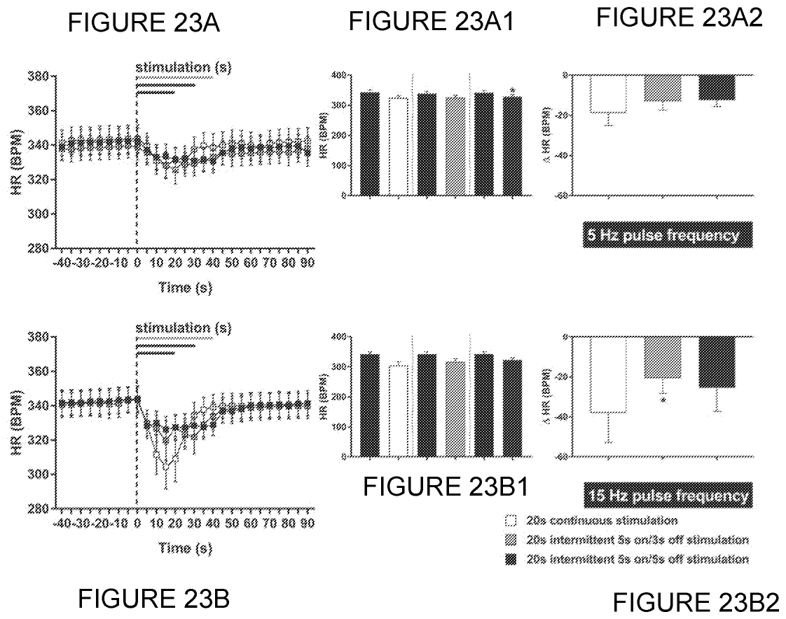

As shown in FIG. 23A-23B2), both continuous and intermittent stimulation produced minor drops in HR, perhaps due to impaired HR baroreflex function in the SHR at this age [95].

Femoral Vascular Resistance (FVR) Responses

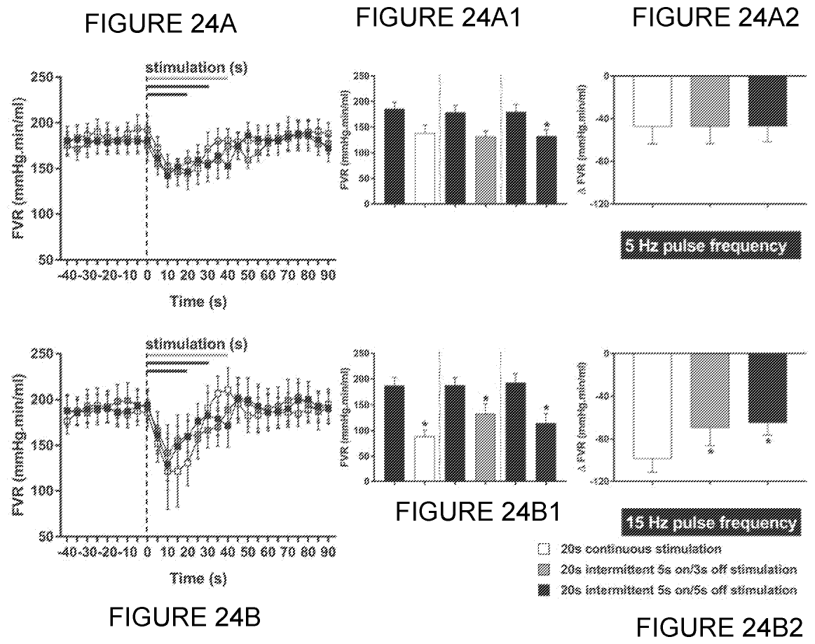

As shown in FIG. 24A (and FIGS. 24A1 and 24A2), low frequency stimulation did not markedly alter reductions in FVR when the ADN was stimulated either continuously or intermittently.

As shown in FIG. 24B (and FIGS. 24B1 and 24B2), high frequency stimulation was associated with greater reductions in FVR; however, intermittent stimulation resulted in a markedly lower drop in FVR relative to the continuous stimulation.

Mesenteric Vascular Resistance (MVR) Responses

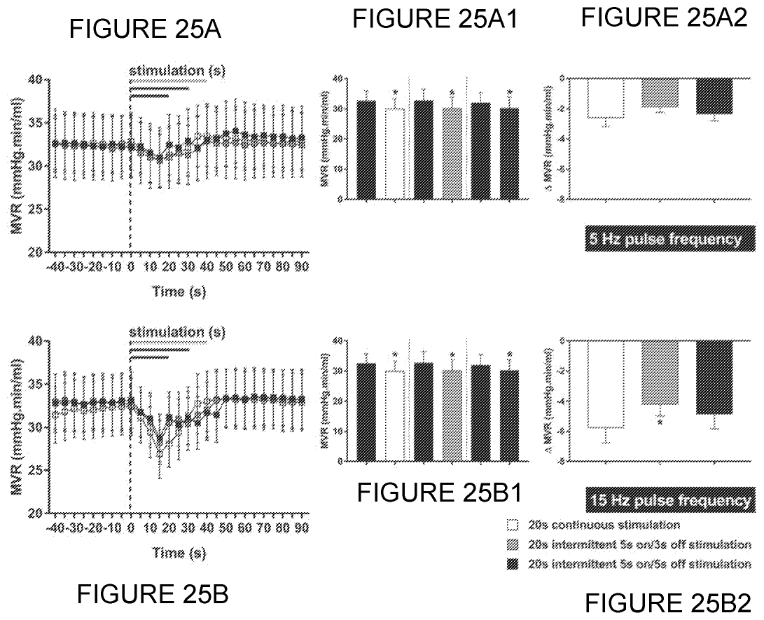

As shown in FIGS. 25A-25B2, both low and high frequency pulses significantly lowered MVR with both continuous and intermittent ADN stimulations. However, bigger reductions in MVR were seen with 15 Hz stimulations.

As shown in FIG. 25B (and FIGS. 25B1 and 25B2), intermittent stimulations at higher frequency had less drastic influence on reductions in MVR compared to continuous stimulation.

Summary

These studies show that low intensity (≤5 Hz) intermittent electrical stimulation is an effective way of modulating the baroreceptor afferents, because it enables low energy consumption for neuromodulation and potentially maintains the integrity of the activated neuronal units.

It was found that low intensity intermittent stimulation of the baroafferent fibers can provide adequate hypotension without drastically altering HR and target organ blood flow and regional vascular resistance. It was considered that, at least under hypertensive conditions, the additive influence of reflex reductions in regional vascular resistance rather than changes in HR may primarily underlie reductions in blood pressure in response to stimulation of the baroreceptor.

Study 8

The cooperativity between the left and right autonomic nerves in influencing the cardiorespiratory profile was investigated.

Studies were performed that compared changes in MAP, heart rate and regional blood flows and vascular resistances elicited by right (R), left (L) or bilateral (LR) electrical stimulation (0.2, 0.5 or 1.0 ms, 5 Hz, 1 mA) of the cervical sympathetic chain (CSC) (8 mm from the SCG) or actually on the superior cervical ganglia (SCG) itself in urethane-anesthetized Sprague-Dawley rats.

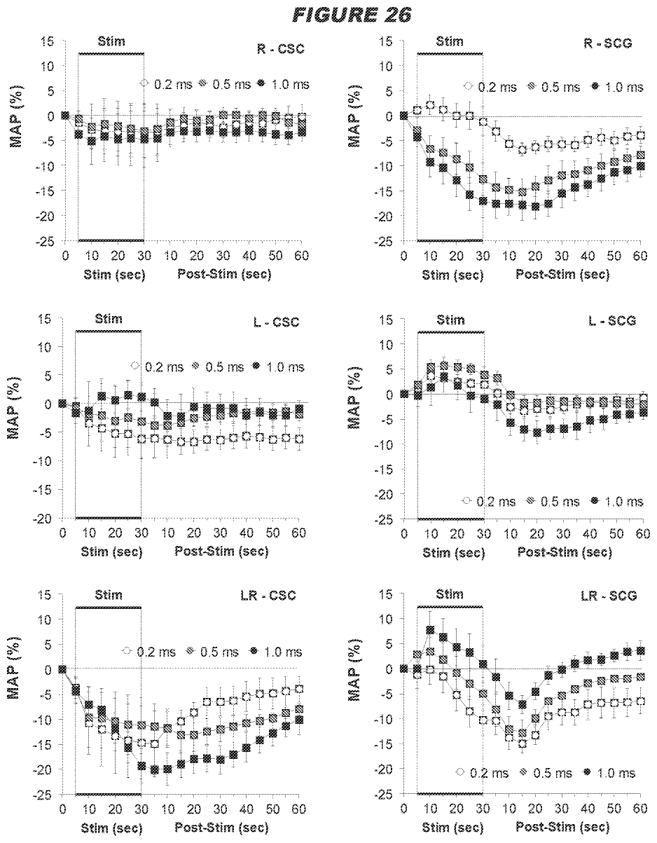

The data from male rats (see FIG. 26) clearly suggests a significant interplay between the CSC and SCG. More specifically, the inventors found evidence for positive cooperativity between the left and right CSC but negative cooperativity between the left and right SCG. The inventors also analyzed the heart rates, and regional vascular resistances with similar profound results.

These data support that simultaneous stimulation of ADN or CSN bilaterally would elicit greater therapeutic cardiorespiratory profiles. There is compelling evidence that centrally-directed inputs from left and right CSN substantially influence one another and there is evidence for both positive and negative cooperativity [96,97,98]. Despite detailed knowledge about the morphology and function of the left and right ADN [99,100,101,102,103], there is no information regarding the possibility that centrally-directed inputs from left or right ADN can influence one another's ability to exert depressor responses.

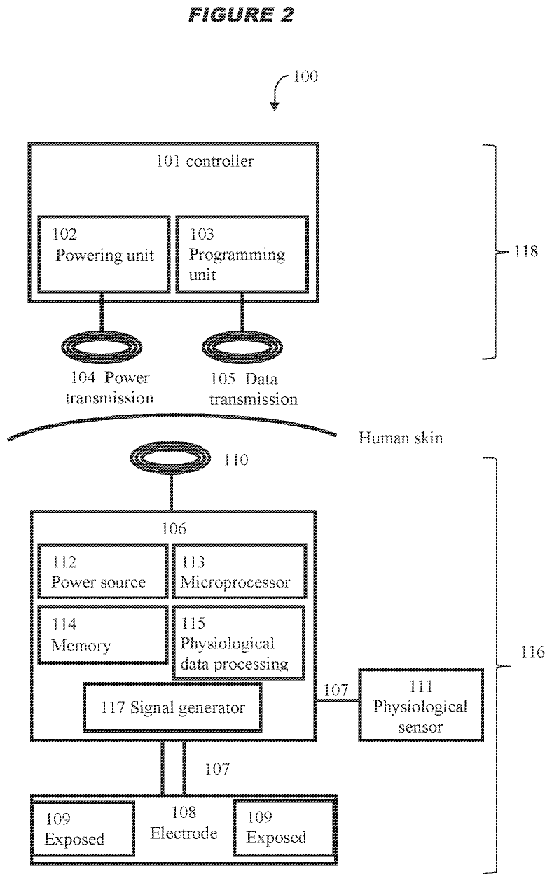

Due to the cross-talk between the baroreceptor activities transmitted by the ADN and the CSN, the inventors consider that simultaneous stimulation of ADN and CSN, especially ipsilateral ADN and CSN stimulation, would elicit greater therapeutic cardiorespiratory profiles. It is unclear as to whether the co-activation of ADN afferents and CSN afferents would promote or inhibit one another's actions. There have been several studies that have addressed this question in various experimental paradigms in dogs [104,105,106,107,108109], cats [110,111,112,113], rabbits [114,115,116,117] and rats [118,119,120,121]. Kendrick et al. [104] demonstrates the existence of a very strong positive cooperativity between the ADN and ipsilateral CSN in dogs, (see FIGS. 2 and 3 in Kendrick et al.).

However, the results from the other studies varied according to stimulation parameters (e.g. pulse-width) and the exact timing of stimuli, with some studies showing a positive cooperativity between the ADN and ipsilateral CSN [104,105,110,112,114,117,121], others showing negative cooperativity [106-109,111,119,120] and others showing no cooperativity (simple summation of inputs) [113,115,116,118].

Study 9

This study aimed to determine differences in cardiovascular responses upon left and right unilateral or bilateral ADN neural modulation in male spontaneously hypertensive (SHR) rats.

Methods

Male spontaneously hypertensive rats (SHR, 335-355 g, 25-27 weeks old) were anaesthetized with 50 mg/kg intraperitoneal injection of sodium pentobarbital and maintained with an intravenous infusion of 10 mg/kg/hr sodium pentobarbital into the right femoral vein. Mean arterial blood pressure (MAP) was measured via an intravenous cannula into the right carotid artery. Heart rate (HR) was derived from the pulsatile signal of mean MAP. A transonic flow probes were placed around the mesenteric and femoral arteries to simultaneously measure regional blood flow and calculate mesenteric (MVR) and femoral (FVR) vascular resistance. Vascular resistance was calculated by the formula: vascular resistance (VR, mmHg·min·ml⁻¹)=mean arterial pressure (MAP, mmHg)/blood flow (BF, ml·min⁻¹). A bipolar electrode was placed around the left and right aortic depressor nerve and stimulation (right, left and bilateral) delivered using a grass stimulator (1, 2.5, 5, 10, 20 and 40 Hz at 0.4 mA, 0.2 ms for 20 s separated by at least 2 minutes). All variables were allowed to return to baseline pre-stimulus levels before the application of the next stimulus.

Results

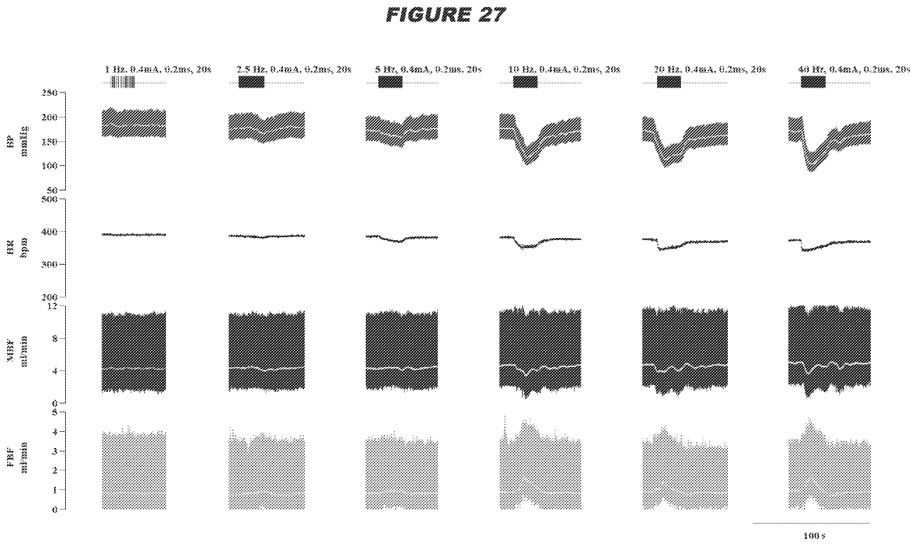

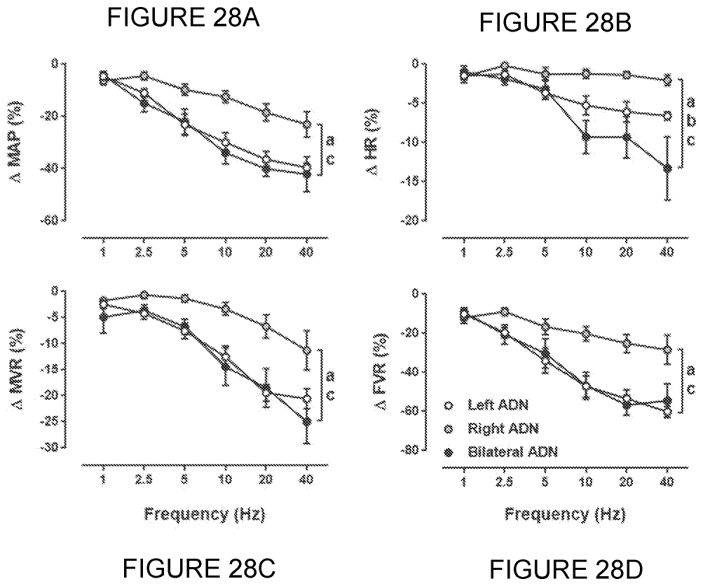

The representative trace in FIG. 27 demonstrates stimulus-dependent changes in blood pressure, heart rate, mesenteric (MBF) and femoral (FBF) blood flows. There were preferential reductions (˜2 folds) in FVR in response to ADN stimulation relative to reductions in MVR. As seen in FIGS. 28A-28D left and bilateral ADN stimulation evoked greater reductions in MAP and HR. This was associated with greater left and bilateral ADN-mediated reductions in both MVR and FVR. Regardless of the side of stimulation ADN-mediated bradycardia was minimal (maximum 15% with bilateral stimulation).

Conclusion

There is preferential central integration of afferent neurotransmission evoked by left aortic baroreceptors, which was evidenced by greater baroreflex-mediated depressor responses relative to activation of the right afferent fibres. Greater reductions in heart rate and vascular resistance evoked by left ADN stimulation likely contribute to the enhanced depressor responses. In SHR males, bilateral ADN stimulation does not produce additive effects on the expression of cardiovascular responses to activation of the baroreceptor afferents and is therefore not superior to left ADN stimulation.

Clinically, this may have implications in fine-tuning the magnitude of baroreflex-driven blood pressure drops in patients in relation to the severity and chronicity of hypertension.

Study 10

This study aimed to determine differences in cardiovascular responses upon left and right unilateral or bilateral ADN neural modulation in male Sprague Dawley rats.

Methods

Male Sprague Dawley (SD) rats (350-460 g, 15-20 weeks old) were anaesthetized with 1.2 g/kg intraperitoneal injection of urethane and maintained with 0.1 ml supplemental intravenous doses of 40% urethane injected into the right femoral vein as required. Mean arterial blood pressure (MAP) was measured via an intravenous cannula into the right femoral artery. Heart rate (HR) was derived from the pulsatile signal of mean MAP. A transonic flow probes were placed around the mesenteric and femoral arteries to simultaneously measure regional blood flow and calculate mesenteric (MVR) and femoral (FVR) vascular resistance. Vascular resistance was calculated by the formula: vascular resistance (VR, mmHg·min·ml⁻¹)=mean arterial pressure (MAP, mmHg)/blood flow (BF, ml·min⁻¹).

A bipolar electrode was placed around the left and right aortic depressor nerve (ADN) and stimulation (right, left and bilateral) delivered using a grass stimulator (1, 2.5, 5, 10, 20 and 40 Hz at 0.4 mA, 0.2 ms for 20 s separated by at least 2 minutes). All variables were allowed to return to baseline pre-stimulus levels before the application of the next stimulus.

Results

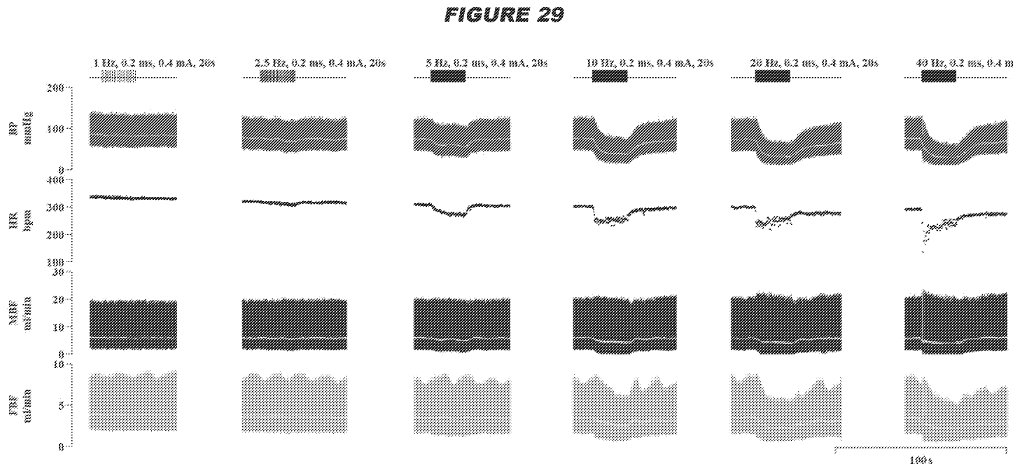

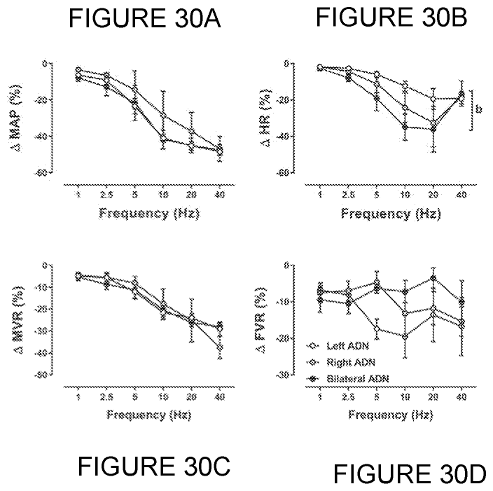

The representative trace in FIG. 29 demonstrates raw changes in blood pressure (BP), heart rate (HR), mesenteric (MBF) and femoral (FBF) blood flows. As seen in FIGS. 30A-30D, irrespective of stimulation side, ADN stimulation resulted in frequency-dependent drops in MAP, HR and MVR. FVR, in contrast, demonstrated a modest frequency-independent decrease of ˜10-20% in response to ADN stimulation (largest drop=20% with left ADN at 20 Hz). MVR reductions in response to ADN stimulation were approximately 40% regardless of the stimulation side and were therefore double that of FVR drops. Reflex depressor responses to left ADN stimulation tended (P=0.06) to be greater than those evoked by stimulation of the right ADN; however, this did not reach statistical significance. Bilateral ADN stimulation was able to evoke comparable drops in HR relative to right ADN stimulation, yet markedly greater drops in HR compared with left ADN stimulation.

Conclusion

The data shows a trend of preferential central integration of afferent neurotransmission evoked by left aortic baroreceptors since baroreflex-triggered depressor responses tended to be relatively greater compared to activation of the right afferent fibres. Despite, the left and right ADN evoking similar effects on MVR and the left ADN evoking a smaller drop in HR than the right ADN, the depressor effect of the left was still greater. Therefore, suggesting that HR and MVR do not underlie the preferential left ADN-mediated drops in blood pressure. The larger reductions in FVR in response to left ADN stimulation, however, may have been responsible for the trended difference in the reflex depressor response.

Study 11

This study aimed to determine differences in cardiovascular responses upon left and right unilateral or bilateral ADN neural modulation in female spontaneously hypersensitive rats.

Methods

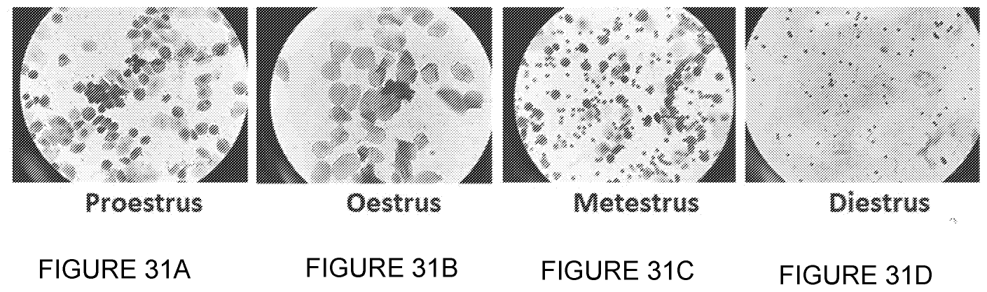

Female spontaneously hypertensive rats (SHR, 185-215 g, 25-29 weeks old) were matched for the diestrus phase of the oestrus cycle (FIGS. 31A-31D) where hormonal variations are minimal. Rats were screened in the morning for at least 2 consecutive cycles (8 days) prior to experimentation. Vaginal secretions were collected with a plastic pipette filled with 20 μL of saline (NaCl 0.9%) by inserting the tip into the rat vagina, but not deeply. Vaginal fluids were then placed on glass slides, fixed, stained (methylene blue, toluidine blue and hematoxylin) and observed under a light microscope to recognize different cell types in the sample. On the day of neurostimulation experiment, rats were anaesthetised with 50 mg/kg intraperitoneal injection of sodium pentobarbital and maintained with an intravenous infusion of 10 mg/kg/hr sodium pentobarbital into the right femoral vein. Mean arterial blood pressure (MAP) was measured via an intravenous cannula into the right femoral artery. Heart rate (HR) was derived from the pulsatile signal of mean MAP. A transonic flow probes were placed around the mesenteric and femoral arteries to simultaneously measure regional blood flow and calculate mesenteric (MVR) and femoral (FVR) vascular resistance. Vascular resistance was calculated by the formula: vascular resistance (VR, mmHg·min·ml-1)=mean arterial pressure (MAP, mmHg)/blood flow (BF, ml·min-1). A bipolar electrode was placed around the left and right aortic depressor nerve (ADN) and stimulation (right, left and bilateral) delivered using a grass stimulator (1, 2.5, 5, 10, 20 and 40 Hz at 0.4 mA, 0.2 ms for 20 s separated by at least 2 minutes). All variables were allowed to return to baseline pre-stimulus levels before the application of the next stimulus.

Results

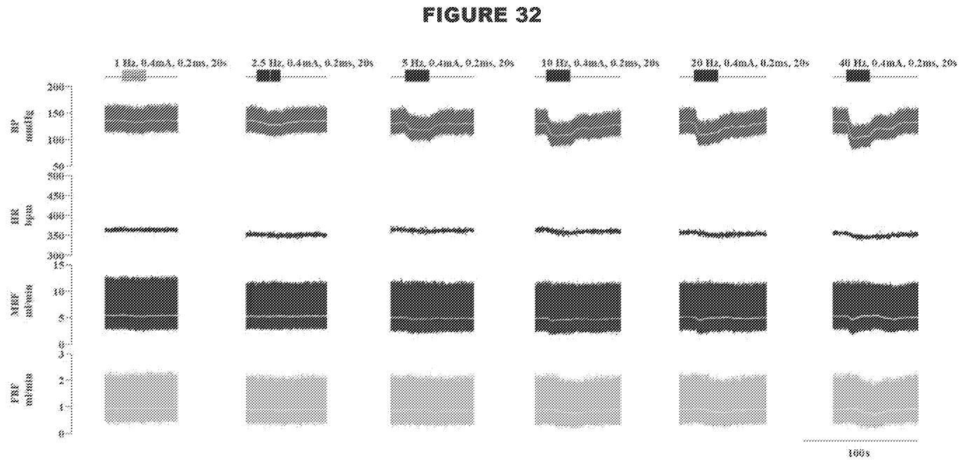

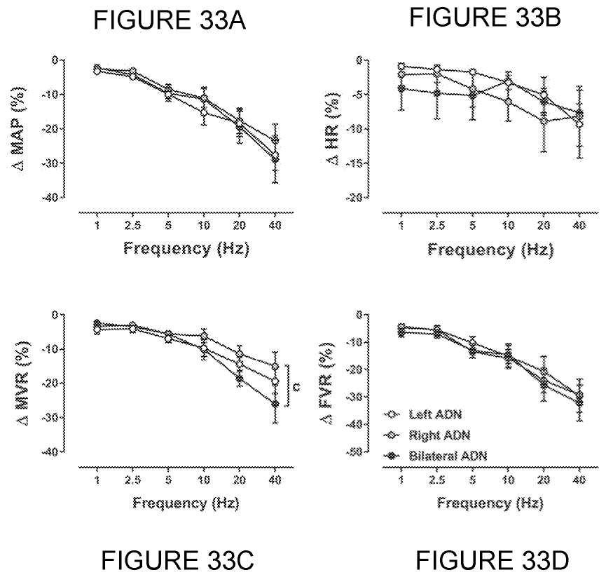

The representative trace in FIG. 32 demonstrates raw changes in blood pressure (BP), heart rate (HR), mesenteric (MBF) and femoral (FBF) blood flows. As seen in FIGS. 33A-33D, with the exception of HR changes, there were frequency-dependent drops in MAP, MVR and FVR. Reflex reductions in MAP, HR and FVR in response to left, right and bilateral ADN stimulation were comparable between groups. Left versus right reductions in MVR were also similar; however, bilateral stimulation evoked greater reductions in MVR relative to the right side stimulation. Regardless of the side of stimulation, ADN-mediated bradycardia was minimal (maximum 10% with bilateral stimulation) and reductions in MVR and FVR were relatively similar (maximum 30% with bilateral stimulation).

Conclusion

Central integration of afferent neurotransmission evoked by left and right aortic baroreceptors is similar in the female SHR. This was evidenced by comparable baroreflex-mediated depressor responses in left versus right ADNs. Similar depressor responses in the left versus right stimulation may have been contributed to by the lack of significant baroreflex-driven changes in HR and vascular resistance. The modest decrease in MVR in response to bilateral stimulation does not seem to significantly impact the reflex depressor response in female SHR. Therefore it is believed that bilateral ADN stimulation does not produce additive effects on the expression of cardiovascular responses to activation of the baroreceptor afferents and is therefore no superior to either left or right ADN stimulation. Clinically, targeting either the left or right aortic nerves in female hypertensive subjects may be able to provide adequate reductions in BP and bilateral stimulation may not contribute any added therapeutic benefit.

Study 12 This study aimed to determine differences in cardiovascular responses upon left and right unilateral or bilateral ADN neural modulation in female Sprague Dawley rats.

Methods

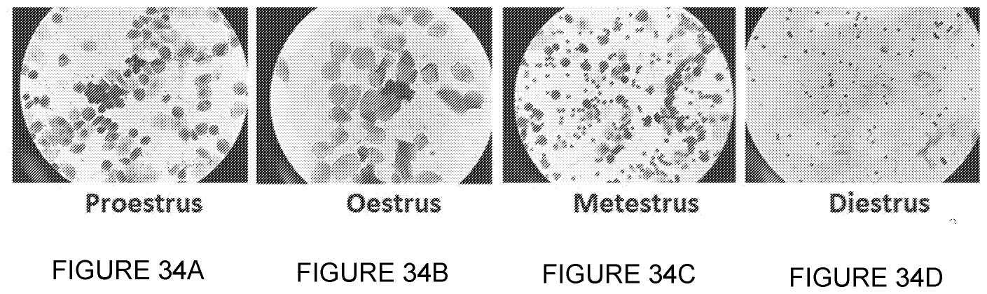

Female Sprague Dawley (SD) rats (222-255 g, 15-18 weeks old) were matched for the diestrus phase of the oestrus cycle (FIGS. 34A-34D) where hormonal variations are minimal. Rats were screened in the morning for at least 2 consecutive cycles (8 days) prior to experimentation. Vaginal secretions were collected with a plastic pipette filled with 20 μL of saline (NaCl 0.9%) by inserting the tip into the rat vagina, but not deeply. Vaginal fluids were then placed on glass slides, fixed, stained (methylene blue, toluidine blue and haematoxylin) and observed under a light microscope to recognize different cell types in the sample. On the day of neurostimulation experiment, rats were anaesthetised with 1.2 g/kg intraperitoneal injection of urethane and maintained with 0.1 ml supplemental intravenous doses of 40% urethane injected into the right femoral vein as required. Mean arterial blood pressure (MAP) was measured via an intravenous cannula into the right femoral artery. Heart rate (HR) was derived from the pulsatile signal of mean MAP. A transonic flow probes were placed around the mesenteric and femoral arteries to simultaneously measure regional blood flow and calculate mesenteric (MVR) and femoral (FVR) vascular resistance. Vascular resistance was calculated by the formula: vascular resistance (VR, mmHg·min·ml-1)=mean arterial pressure (MAP, mmHg)/blood flow (BF, ml·min−1). A bipolar electrode was placed around the left and right aortic depressor nerve (ADN) and stimulation (right, left and bilateral) delivered using a grass stimulator (1, 2.5, 5, 10, 20 and 40 Hz at 0.4 mA, 0.2 ms for 20 s separated by at least 2 minutes). All variables were allowed to return to baseline pre-stimulus levels before the application of the next stimulus.

Results

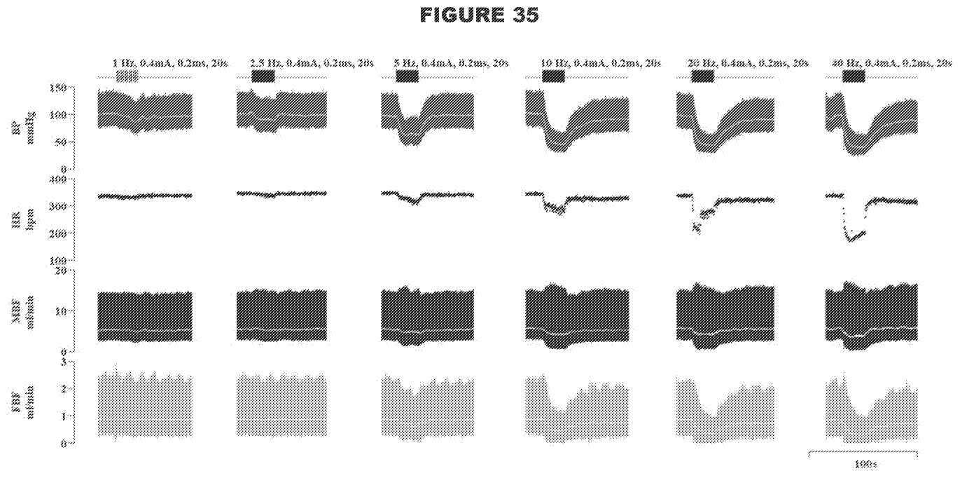

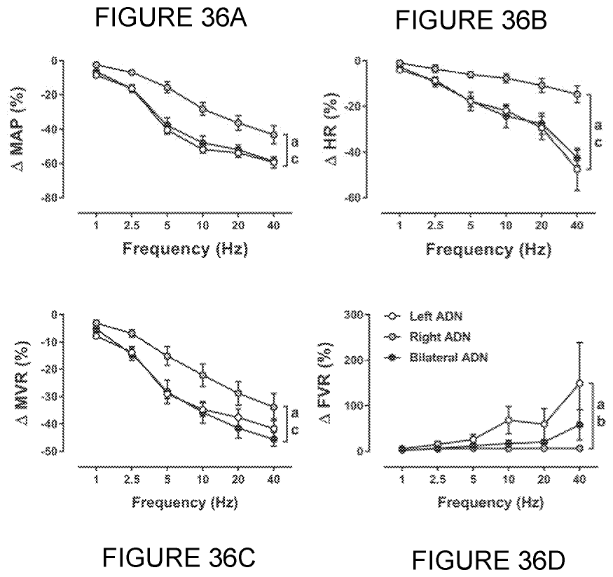

The representative trace in FIG. 35 demonstrates frequency-dependent changes in blood pressure (BP), heart rate (HR), mesenteric (MBF) and femoral (FBF) blood flows with ADN stimulation. As seen in FIGS. 36A-36D, irrespective of stimulation side, ADN stimulation resulted in frequency-dependent drops in MAP, HR and MVR. FVR, in contrast, demonstrated a biphasic response consisting of a modest decrease of ˜15-20% in response to ADN stimulation (data not shown) followed by a frequency-dependent increase. Left and bilateral ADN stimulation evoked greater reductions in MAP, HR and MVR relative to right ADN stimulation. Secondary increases in FVR in response to left ADN stimulation were markedly greater compared with both right and bilateral ADN stimulation.

Conclusion

There is preferential central integration of afferent neurotransmission evoked by left aortic baroreceptors, which was evidenced by greater baroreflex-mediated depressor responses relative to activation of the right afferent fibres. Greater reductions in HR and vascular resistance evoked by left ADN stimulation likely contribute to the enhanced depressor responses. The secondary increase in FVR in response to ADN stimulation may represent a compensatory mechanism coming into play to counteract the marked drop in blood pressure in response to baroreflex activation. In SD females, bilateral ADN stimulation does not produce additive effects on the expression of cardiovascular responses to activation of the baroreceptor afferents and is therefore no superior to left ADN stimulation.

### GENERAL CONCLUSION

These data demonstrate that the application of an electrical signal to modulate a subject's ADN and/or the CSN provides a useful way for treating or preventing cardiovascular disorders and disorders associated therewith. The application is particularly effective with low intensity (e.g. ≤10 Hz) intermittent stimulation (e.g. 5 s on; 3 s or 5 s off; for 20 s). The application is also particularly effective when the neural activity of both the ADN and CSN are modulated (e.g. stimulated) because of the cooperativity between these nerves, especially between ipsilateral ADN and CSN afferents.

### REFERENCES

[1] Lau et al., Front Physiol. 7: 384, 2016.
[2] Salman et al., Curr Hypertens Rep. 18: 18, 2016.
[3] Thrasher et al., Am J Physiol Regul Integr Comp Physiol. 288: R819-R827, 2005.
[4] Chapleau et al., Clin Auton Res. 13: 310-313, 2003.
[5] Dampney et al., Clin Exp Pharmacol Physiol. 29: 261-268, 2002.
[6] Machado et al., Ann N Y Acad Sci. 940: 179-196, 2001.
[7] Head et al., J Cardiovasc Pharmacol. 26 Suppl 2: S7-S16, 1995.
[8] Spyer et al., Rev Physiol Biochem Pharmacol. 88: 24-124, 1981.
[9] Abuzinadah et al., Clin Auton Res. 26: 465-466, 2016.
[10] van de Vooren et al., J Appl Physiol (1985). 102: 1348-1356, 2007.
[11] Silveira et al., Sleep. 31: 328-333, 2008.
[12] Hohage et al., Med Klin (Munich). 95: 254-260, 2000.
[13] Irigoyen et al., Braz J Med Biol Res. 21: 869-872, 1988.
[14] Padilha et al., Clin Exp Hypertens A. 10 Suppl 1: 123-129, 1988.
[15] Jungeira et al., J Physiol. 259: 725-735, 1976.
[16] Trindade et al., Braz J Med Biol Res. 17: 209-217, 1984.
[17] Possas et al., Am J Physiol Regul Integr Comp Physiol. 290: R741-R748, 2006.
[18] Brognara et al., Life Sci. 148: 99-105, 2016.
[19] Turner et al., Life Sci. 106: 40-49, 2014.
[20] Durand et al., Braz J Med Biol Res. 45: 444-449, 2012.
[21] Durand Mde et al., Am J Physiol Regul Integr Comp Physiol. 300: R418-R427, 2011.
[22] Durand et al., Braz J Med Biol Res. 42: 53-60, 2009.
[23] Salgado et al., Am J Physiol Heart Circ Physiol. 292: H593-H600, 2007.
[24] De Paula et al., Am J Physiol. 277: R31-R38, 1999.
[25] Crill et al., Am J Physiol. 214: 269-276, 1968.
[26] Hildebrandt, Exp Neurol. 45: 590-605, 1974.
[27] Kirchheim, Physiol Rev. 56: 100-177, 1976.
[28] Ciriello et al., J Auton Nerv Syst. 3: 299-310, 1981.
[29] Ciriello et al., J Auton Nerv Syst. 1: 13-32, 1979.
[30] Schneider et al., Naunyn Schmiedebergs Arch Pharmacol. 352: 291-296, 1995.
[31] Cechetto D F et al., Am J Physiol. 244: R646-R651, 1983.
[32] Chapleau et al., Clin Exp Pharmacol Physiol Suppl. 15: 31-43, 1989.
[33] Undesser et al., Am J Physiol. 246: H302-H305, 1984.
[34] Gonzalez et al., Hypertension 5: 346-352, 1983.
[35] Santa Cruz Chavez et al., Am J Physiol Heart Circ Physiol. 307: H910-H921, 2014.
[36] Zhang et al., Neurosci Lett. 604: 1-6, 2015.
[37] Liao et al., PLoS One 9: e109313, 2014.
[38] Liao et al., J Cardiovasc Pharmacol. 64: 431-437, 2014.
[39] Mostarda et al., J Card Fail. 17: 519-512, 2011.
[40] Sheng et al., J Interv Card Electrophysiol. 45: 131-140, 2016.
[41] Love et al., Acta Neuropathol. 131: 645-658, 2016.
[42] Di Marco et al., Neurobiol Dis. 82: 593-606, 2015.
[43] Nelson et al., Biochim Biophys Acta. 1862: 887-900, 2016.
[44] Meel-van den Abeelen et al., Neurobiol Aging. 34: 1170-1176, 2013.
[45] Kaufmann et al., Semin Neurol. 23: 351-363, 2003.
[46] Laosiripisan et al., Clin Auton Res. 25: 213-218, 2015.
[47] Tarumi et al., Neuroimage 110: 162-170, 2015.
[48] Femminella et al., J Alzheimers Dis. 42: 369-377, 2014.
[49] Claassen et al., J Alzheimers Dis. 17: 621-629, 2009.
[50] Sabayan et al., Ageing Res Rev. 11: 271-277, 2012.
[51] Szili-Török et al., Neurobiol Aging 22: 435-438, 2001.
[52] Schoenborn et al., Folia Biol (Krakow). 44: 123-129, 1996.
[53] Horne et al., Am J Physiol. 260: H1283-H1289, 1991.
[54] Horne et al., Am J Physiol. 256: H434-H440, 1989.
[55] Dworkin et al., AmJ Physiol Regul Integr Comp Physiol. 286: R597-R605, 2004.
[56] Jungeira et al., J Physiol. 259: 725-735, 1976.
[57] Hofer et al., Sleep 8: 40-48, 1985.
[58] Padilha et al., J Cardiovasc Pharmacol. 10 (Suppl 12): S194-S198, 1987.
[59] Sei et al., J Sleep Res. 8: 45-50, 1999.
[60] Silveira et al., Sleep 31: 328-333, 2008.
[61] Mancia et al., Cir. Res. 1983, 53(1): 96-104.
[62] Germano et al., 1984, Clin. Cardiol. 7, 525-535.
[63] Randich et al., Ann N Y Acad Sci. 467: 385-401, 1986.
[64] Sdvoz-Couche et al., Pain 99: 71-81, 2002.
[65] Meller et al., Hypertension 15: 797-802, 1990.
[66] Vallbo et al. Physiological Reviews 1979; 59, 919-957.
[67] Macefield et al. The Journal of Physiology (London) 1994; 481, 799-809.
[68] Esler et al. Hypertension, 1988; 11, 3-20.
[69] Brown et al., Journal of Physiology 1975; 138, 81-102.
[70] Grassi et al., M. Journal of Hypertension, 1999; 17, 719-734.
[71] US 2015/0174397.
[72] US 2012/0035679.
[73] US 2014/0180391.
[74] US 2006/0004430.
[75] Toorop et al., J. Vase. Surg. 50(1): 177-182.
[76] Abboud F M. Am J Cardiol. 44: 903-911, 1979.
[77] Lacolley et al., Neuroscience 143: 289-308, 2006.
[78] Ohta et al., Genetic Hypertens. 218: 61-63, 1992.
[79] De Paula et al., Am J Physiol. 277: R31-R38, 1999.
[80] Possas et al., Am J Physiol. 290: R741-R748, 2006.
[81] Salgado et al., Am J Physiol Physiol. 292: H593-600, 2007.
[82] Durand et al., Braz J Med Biol Res. 45: 444-449, 2012.
[83] Sapru et al., Am J Physiol. 230: 664-674, 1976.
[84] Andresen et al., Circ Res. 43: 728-738, 1978.
[85] Andresen et al., Am J Physiol. 256: H1228-H1235, 1989.
[86] Widdop et al., J Hypertension 8: 269-275, 1990.
[87] Fazan et al., J Auton Nerv Syst. 77: 133-139, 1999.
[88] Henry et al., Hypertension 16: 422-428, 1990.
[89] van den Buuse M. et al., Physiol Behav. 55: 783-787, 1994.
[90] Oosting et al., J Hypertens. 15: 401-410, 1997.
[91] Head et al., J Hypertens. 22: 2075-2085, 2004.
[92] Snitsarev et al., Auton Neurosci. 98: 59-63, 2002.
[93] Drummond et al., Ann N Y Acad Sci. 940: 42-47, 2001.
[94] Drummond et al., Neuron 21: 1435-1441, 1998.
[95] Widdop et al., 1990, J. Hypertens, 8(3): 269-75.
[96] Kawada et al., Am J Physiol. 277: H857-H865, 1999.
[97] Felder et al., Am J Physiol. 253: H1127-H1135, 1987.
[98] Greene et al., Am J Physiol. 250: H96-H107, 1986.
[99] Fazan et al., J Auton Nerv Syst. 77: 133-139, 1999.
[100] Douglas et al., J Physiol. 134: 167-178, 1956.
[101] Douglas et al., J Physiol. 132: 187-198, 1956.
[102] Santa Cruz Chavez et al., Am J Physiol Heart Circ Physiol. 307: H910-H921, 2014.
[103] Peters et al., J Auton Nerv Syst. 27: 193-205, 1989.
[104] Kendrick et al., Am J Physiol. 236: H127-H133, 1979.
[105] Kendrick et al., Am J Physiol. 237: H662-H667, 1979.
[106] Pisarri et al., Am J Physiol. 243: H607-H613, 1982.
[107] Brunner et al., Circ Res. 55: 740-750, 1984.
[108] Lalley et al., Proc Soc Exp Biol Med. 176: 384-391, 1984.
[109] Heitz et al., Proc Soc Exp Biol Med. 143: 854-857, 1973.
[110] Oberg et al., Acta Physiol Scand. 113: 129-137, 1981.
[111] Chruscielewski et al., Acta Neurobiol Exp (Wars). 41: 175-187, 1981.
[112] Szulczyk et al., Acta Neurobiol Exp (Wars). 31: 15-25, 1977.
[113] Szulczyk et al., J Auton Nerv Syst. 2: 355-364, 1980.
[114] Ishikawa et al., Circ Res. 52: 401-410, 1983.
[115] Thames et al., Am J Physiol. 246: H851-H857, 1984.
[116] Yamazaki et al., Am J Physiol. 257: H465-H472, 1989.
[117] Hayward et al., Am J Physiol. 264: H1215-H1222, 1993.
[118] Kawada et al., Am J Physiol Regul Integr Comp Physiol. 312: R787-R796, 2017.
[119] Murata et al., Exp Physiol. 84: 897-906, 1999.
[120] Kongo et al., Exp Physiol. 84: 47-56, 1999.
[121] Fan et al., Am J Physiol. 271: H2218-H2227, 1996.

1. A system for modulating neural activity in a subject's aortic depressor nerve (ADN) and/or carotid sinus nerve (CSN), the system comprising:
at least one neural interfacing element having at least one electrode configured to be in signaling contact with the ADN and/or CSN that is being modulated, and
a signal generator comprising at least one voltage or current source configured to generate at least one signal to be applied to the ADN and/or CSN that is being modulated via the at least one electrode to modulate the neural activity of the ADN and/or CSN that is being modulated to produce a change in a physiological parameter in the subject,
wherein the amplitude of the at least one signal is ≤0.4 mA;
wherein the change in the physiological parameter is one or more of the group consisting of: a decrease in mean arterial pressure, a decrease in heart rate, an increase in minute ventilation, an improvement in regularity of a heart rhythm, an improvement in heart conduction, an increase in heart contractility, a decrease in vascular resistance, an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception;
wherein the signal generator is controlled to deliver to the ADN and/or CSN that is being modulated the at least one signal having a total intensity, the total intensity being below a predetermined threshold, the predetermined threshold defined as the total intensity of a signal required to be received by the ADN and/or CSN to produce a ≤30 mmHg drop in the mean arterial blood pressure.

2. The system of claim 1, wherein the at least one signal is configured to be applied to the ADN, and the at least one electrode is suitable for placement on or around the ADN.

3. The system of claim 1, wherein the at least one signal is configured to be applied to the CSN, wherein the at least one electrode is suitable for placement on or around the CSN.

4. The system of claim 1, wherein the at least one signal includes a first signal and a second signal, the first signal is configured to be applied to the ADN and the second signal is configured to be applied to the CSN, wherein the at least one electrode includes a first electrode and a second electrode, the first electrode is suitable for placement on or around the ADN and the second electrode is suitable for placement on or around the CSN, wherein the first signal is to be applied via the first electrode and the second signal is to be applied via the second electrode.

5. The system of claim 1, wherein the at least one signal is configured to be applied to the ADN and/or CSN unilaterally or bilaterally.

6. The system of claim 4, wherein the at least one signal is configured to be applied to the ADN and the CSN ipsilaterally.

7. The system of claim 1, wherein the predetermined threshold is ≤30 μAs.

8. The system of claim 1, wherein the total intensity of the signal is between 0.1 TINT and 0.9 TINT, where TINT is the predetermined threshold.

9. The system of claim 1, wherein the at least one signal has a predetermined duty cycle of ≤65%.

10. The system of claim 1, wherein the at least one signal has a pulse width of ≤1 ms.

11. The system of claim 1, wherein the frequency of the at least one signal is ≤70 Hz.

12. The system of claim 1, wherein the at least one signal is applied in a (ONy−OFFz)n pattern where n>1, y>0, and z>0, and the at least one signal is applied for: (a) ≤20 s, or (b) ≤30 min at any given time up to 12 times a day.

13. The system of claim 1, wherein the signal generator is controlled to deliver the at least one signal during a specific time of a day.

14. The system of claim 1, further comprising a detector configured to:
detect one or more signals indicative of one or more second physiological parameters;
determine from the one or more signals the one or more second physiological parameters;
determine the one or more second physiological parameters indicative of worsening of the second physiological parameter; and cause the at least one signal to be applied to the ADN and/or CSN via the at least one electrode, wherein the second physiological parameter is one or more of the group consisting of: systemic arterial blood pressure, heart rate, heart rhythm, electrical conduction in the heart and heart contractility, vascular resistance, cardiac output, rate of blood flow, minute ventilation, and pain perception.

15. The system of claim 14, further comprising a memory arranged to store data pertaining to the second physiological parameters indicative of a disorder associated with malfunction or loss of the baroreceptor reflex, wherein determining the one or more second physiological parameters indicative of worsening of the second physiological parameter comprises comparing the one or more second physiological parameters with the data.

16. The system of claim 14, wherein one of the second physiological parameters is the arterial blood pressure, wherein the detector comprises a pressure sensor.

17. A method of treating or preventing a disorder associated with malfunction or loss of a baroreceptor reflex in a subject by reversibly modulating neural activity of a subject's aortic depressor nerve (ADN) and/or carotid sinus nerve (CSN), comprising: implanting in the subject the system of claim 1; positioning the neural interfacing element in signaling contact with the ADN and/or CSN; and optionally activating the system to provide at least one signal wherein the at least one signal is applied to the ADN and/or the CSN.

18. The method of claim 17, wherein the method is for treating or preventing a cardiovascular disorder and a disorder associated therewith, or a cardiorespiratory and a disorder associated therewith.

19. A method for treating or preventing a disorder associated with malfunction or loss of a baroreceptor reflex, comprising:
applying a signal to a subject's aortic depressor nerve (ADN) and/or carotid sinus nerve (CSN) via at least one neural interfacing element having at least one electrode in signaling contact with the ADN and/or CSN, such that the signal reversibly modulates neural activity of the ADN and/or CSN to produce a change in a physiological parameter in the subject:
wherein the change in the physiological parameter is one or more of the group consisting of: a decrease in mean arterial pressure, a decrease in heart rate, an increase in minute ventilation, an improvement in the regularity of the a heart rhythm, an improvement in heart conduction, an increase in heart contractility, a decrease in vascular resistance, an increase in cardiac output, an increase in blood flow, an increase in minute ventilation, an increase in a hemodynamic response, a decrease in a chronotropic evoked response, a decrease in a dromotropic evoked response, a decrease in a lusitropic evoked response, a decrease in an inotropic evoked response, and a decrease in pain perception,
wherein the total intensity of the signal received by the ADN and/or CSN that is being modulated is below a predetermined threshold, the predetermined threshold defined as the total intensity of a signal required to be received by the ADN and/or CSN to produce a ≤30 mmHg drop in the mean arterial blood pressure: and wherein the amplitude of the at least one signal is ≤0.4 mA.
